(19) United States
(12) Patent Application Publication
(10) Pub. No.: US 2016/0061841 A1
(43) Pub. Date: Mar. 3, 2016

(54) NRF2-BASED CANCER TREATMENT AND DETECTION METHODS AND USES

(71) Applicant: CanBas Co., Ltd., Numazu (JP)

(72) Inventors: Naoki MINE, Shizuoka (JP); Takumi KAWABE, Shizuoka (JP)

(21) Appl. No.: 14/719,088

(22) Filed: May 21, 2015

Related U.S. Application Data
(60) Provisional application No. 62/002,076, filed on May 22, 2014.
(60) Provisional application No. 62/036,476, filed on Aug. 12, 2014.

(51) Int. Cl. G01N 33/574; A61K 38/10; C12Q 1/68

(52) U.S. Cl. CPC G01N 33/57484; C12Q 1/6886; A61K 38/10; C12Q 2600/158; C12Q 2600/156

(57) ABSTRACT

Disclosed herein, inter alia, are methods and uses for treating a cancer in a subject. In various embodiments, a method or use includes measuring expression of nuclear factor erythroid-2 related factor 2 (NRF2), or an NRF2 target gene, in a candidate subject having cancer, or a cancer sample from the candidate subject, and determining the amount of NRF2 in the sample or in the subject having cancer, then comparing the amount of NRF2 determined, or NRF2 target gene determined, to a baseline or reference amount of NRF2 or NRF2 target gene. If the amount of NRF2 or NRF2 target gene in the sample or in the subject having cancer is less than the baseline or reference amount of NRF2 or NRF2 target gene, the subject having the cancer may be or is treated with a peptide or peptidomimetic sequence set forth herein, such as P1, P2, P3, P4, P5, P6 (SEQ ID NO:1) or P6, P5, P4, P3, P2, P1 (SEQ ID NO:2), e.g., CBP501.

### RELATED PATENT APPLICATION

This patent application claims the benefit of U.S. Provisional Application Nos 62/002,076 filed May 22, 2014 and 62/036,476, Aug. 12, 2014, which are incorporated herein by reference in their entirety.

### INTRODUCTION

The cell cycle comprises S phase (DNA replication), M phase (mitosis), and two gap phases (G1 and G2 phases) between S and M phases. Checkpoints in the cell cycle ensure accurate progression, such as monitoring the state of DNA integrity, DNA replication, cell size, and the surrounding environment (Maller, J. L. Curr. Opin. Cell Biol., 3:26 (1991)). It is especially important for multi-cellular organisms to maintain integrity of genome, and there are multiple checkpoints that monitor the state of genome. Among them are G1 and G2 checkpoints existing before DNA replication and mitosis, respectively. It is also important to correct DNA damage before entering S phase, because once damaged DNA is replicated it often gives rise to mutations (Hartwell, L. Cell, 71: 543 (1992)). Progression through G1 and G2 checkpoints without repairing extensive DNA damage induces apoptosis and/or catastrophe.

Most cancer cells carry abnormalities in G1 checkpoint-related proteins such as p53, Rb, MDM-2, p16INK4 and p19ARF (Levine, A. J. Cell, 88:323 (1997)). Alternatively, mutations can cause over-expression and/or over activation of oncogene products, e.g., Ras, MDM-2 and cyclin D, which reduce the stringency of G1 checkpoint. In addition to these mutations, excessive growth factor signaling can be caused by the over expression of growth factors and can reduce the stringency of G1 checkpoint. Together with loss and gain-of-function mutations, continuous activation of growth factor receptors or downstream signal-transducing molecules can cause cell transformation by overriding the G1 checkpoint. Abrogated G1 checkpoint contributes to higher mutation rates and the many mutations observed in cancer cells. As a result, most cancer cells depend on G2 checkpoint for survival against excessive DNA damage (O'Connor and Fan, Prog. Cell Cycle Res., 2:165 (1996)).

The mechanism that promotes the cell cycle G2 arrest after DNA damage is believed to be conserved among species from yeast to human. In the presence of damaged DNA, Cdc2/Cyclin B kinase is kept inactive because of inhibitory phosphorylation of threonine-14 and tyrosine-15 residues on Cdc2 kinase or the protein level of Cyclin B is reduced. At the onset of mitosis, the dual phosphatase Cdc25 removes these inhibitory phosphates and thereby activates Cdc2/Cyclin B kinase. The activation of Cdc2/Cyclin B is equivalent to the onset of M phase.

In fission yeast, the protein kinase Chk1 is required for the cell cycle arrest in response to damaged DNA. Chk1 kinase acts downstream of several rad gene products and is modified by the phosphorylation upon DNA damage. The kinases Rad53 of budding yeast and Cds1 of fission yeast are known to conduct signals from unreplicated DNA. It appears that there is some redundancy between Chk1 and Cds1 because elimination of both Chk1 and Cds1 culminated in disruption of the G2 arrest induced by damaged DNA. Interestingly, both Chk1 and Cds1 phosphorylate Cdc25 and promote Rad24 binding to Cdc25, which sequesters Cdc25 to cytosol and prevents Cdc2/Cyclin B activation. Therefore Cdc25 appears to be a common target of these kinases implying that this molecule is an indispensable factor in the G2 checkpoint.

In humans, both hChk1, a human homologue of fission yeast Chk1, and Chk2/HuCds1, a human homologue of the budding yeast Rad53 and fission yeast Cds1, phosphorylate Cdc25C at serine-216, a critical regulatory site, in response to DNA damage. This phosphorylation creates a binding site for small acidic proteins 14-3-3s, human homologues of Rad24 and Rad25 of fission yeast. The regulatory role of this phosphorylation was clearly indicated by the fact that substitution of serine-216 to alanine on Cdc25C disrupted cell cycle G2 arrest in human cells. However, the mechanism of G2 checkpoint is not fully understood.

### SUMMARY

Disclosed herein, in various embodiments, are methods for treating a cancer in a subject. In one embodiment, a method comprises a) measuring expression of nuclear factor erythroid-2 related factor 2 (NRF2), or an NRF2 target gene, in a candidate subject having cancer, or a cancer sample from the candidate subject, and determining the amount of NRF2 in the sample or in the subject having cancer, b) comparing the amount of NRF2 determined, or NRF2 target gene determined, to a baseline or reference amount of NRF2 or NRF2 target gene, thereby determining if the amount of NRF2 or NRF2 target gene in the sample or in the subject having cancer is less than the baseline or reference amount of NRF2 or NRF2 target gene; and c) treating the subject having the cancer with P1, P2, P3, P4, P5, P6 or P6, P5, P4, P3, P2, P1 (e.g., CBP501) if expression of the NRF2, or the NRF2 target gene in the sample or in the subject having cancer is less than the baseline or reference amount of NRF2 or NRF2 target gene. In another embodiment, a method comprises a) screening for a normal or a functional KEAP 1, or a mutation that reduces or decreases activity, function or expression of KEAP 1 in a candidate subject having cancer, or a cancer sample from the candidate subject, and determining the presence of a normal or a functional KEAP 1 or a mutation that reduces or decreases activity, function or expression of KEAP 1; and b) treating the subject having the cancer with P1, P2, P3, P4, P5, P6 or P6, P5, P4, P3, P2, P1 (e.g., CBP501) if the subject has a normal or a functional KEAP 1 or does not have a mutation that reduces or decreases activity, function or expression of KEAP 1.

Disclosed herein, in various embodiments, are methods for treating a cancer in a subject. In one embodiment, a method comprises a) identifying and/or selecting a subject with a cancer in which NRF2 expression or NRF2 target gene expression in the subject is less than a baseline or reference amount of NRF2 or NRF2 target gene, or where the subject has a normal or a functional KEAP 1 or lacks a mutation that reduces or decreases activity, function or expression of KEAP 1 and b) treating the cancer in the subject with a peptide or peptidomimetic set forth herein (e.g., CBP501) as if the NRF2 or the NRF2 target gene in the sample or in the subject is less than the baseline or reference amount of NRF2 or NRF2 target gene, or if the subject has a normal or a functional KEAP 1 or does not have a mutation that reduces or decreases activity, function or expression of KEAP 1. In another embodiment, a method comprises, a) measuring expression of nuclear factor erythroid-2 related factor 2 (NRF2), or an NRF2 target gene, in a candidate subject having cancer, or a cancer sample from the candidate subject, and determining the amount of NRF2 or NRF2 target gene in the subject or cancer sample, b) comparing the amount of NRF2 determined, or NRF2 target gene determined, to a baseline or reference amount of NRF2 or NRF2 target gene, thereby determining if the amount of NRF2 or NRF2 target gene is less than the baseline or reference amount of NRF2 or NRF2 target gene and c) selecting the subject for cancer treatment with a peptide or peptidomimetic as set forth herein (e.g., CBP501) if the NRF2 or NRF2 target gene in the sample or in the subject is less than the baseline or reference amount of NRF2 or NRF2 target gene.

Further disclosed herein are methods for selecting and/or identifying a subject for cancer treatment with a peptide or peptidomimetic as set forth herein (e.g., CBP501). In one embodiment, a method includes a) screening for a normal or a functional KEAP 1 or a mutation that reduces or decreases activity, function or expression of KEAP 1 and b) selecting the subject for cancer treatment with a peptide or peptidomimetic as set forth herein (e.g., CBP501) if normal or a functional KEAP 1 is present or a mutation that reduces or decreases activity, function or expression of KEAP 1 is absent or not present. In another embodiment, a method includes a) measuring expression of nuclear factor erythroid-2 related factor 2 (NRF2) or NRF2 target gene in a candidate subject having cancer, or a cancer sample from the candidate subject, and determining the amount of NRF2 or NRF2 target gene in the subject or cancer sample, b) comparing the amount of NRF2 determined, or NRF2 target gene determined, to a baseline or reference amount of NRF2 in order to determine if the amount of NRF2 or NRF2 target gene is less than the baseline or reference amount of NRF2 or NRF2 target gene and c) identifying the subject as a candidate for cancer treatment with a peptide or peptidomimetic as set forth herein (e.g., CBP501) if the NRF2 or NRF2 target gene in the sample or in the subject is less than the baseline or reference amount of NRF2 or NRF2 target gene.

In embodiments for screening or for identifying a candidate subject for cancer treatment with a peptide or peptidomimetic as set forth herein (e.g., CBP501), certain aspects can sometimes comprise, a) screening for a normal or a functional KEAP 1 or a mutation that reduces or decreases activity, function or expression of KEAP 1 and b) identifying the subject as a candidate for cancer treatment with a peptide or peptidomimetic as set forth herein (e.g., CBP501) if the normal or a functional KEAP 1 is present, or a mutation mutation that reduces or decreases activity, function or expression of KEAP 1 is absent or not present.

Additionally disclosed herein are methods for characterizing a cancer as more responsive or less responsive to treatment with a peptide or peptidomimetic as set forth herein (e.g., CBP501). In one embodiment, a method includes a) measuring expression of nuclear factor erythroid-2 related factor 2 (NRF2) or NRF2 target gene of cells of a cancer, and determining the amount of NRF2 or NRF2 target gene expressed, b) comparing the amount of NRF2 determined, or NRF2 target gene determined, to a predetermined value for NRF2 or NRF2 target gene in order to determine if the amount of NRF2 or NRF2 target gene is less or greater than the predetermined value for NRF2 or NRF2 target gene and c) characterizing the cancer as more responsive or less responsive to treatment with a peptide or peptidomimetic as set forth herein (e.g., CBP501) if the NRF2 or NRF2 target gene expression is less than or greater than the predetermined value for NRF2 or NRF2 target gene. In another embodiment, a method includes a) screening for a normal or a functional KEAP 1 or a mutation that reduces or decreases activity, function or expression of KEAP 1 and/or b) characterizing the cancer as more responsive or less responsive to treatment with a peptide or peptidomimetic as set forth herein (e.g., CBP501) if the normal or a functional KEAP 1 is present or absent, or a mutation that reduces or decreases activity, function or expression of KEAP 1 is absent or present.

In various embodiments a NRF2 target gene is any of Glutathione reductase (GSR), Glucose-6-phosphate dehydrogenase (G6PD), ATP-binding cassette sub-familyC member2 (ABCC2), Aldo-keto reductase family1C1 (AKR1C1), Aldo-keto reductase family1C3 (AKR1C3), NAD(P)H dehydrogenase, quinonel (NQO1), AKR1B10, γ-glutamyl cysteine synthetase modifier subunit (γGCSm) and/or Glutathione peroxidasel (GPX1).

In some embodiments a baseline or reference level or predetermined value is determined by expression in cancer cells responsive to a peptide or peptidomimetic as set forth herein (e.g., CBP501) treatment compared to cancer cells less-responsive to a peptide or peptidomimetic (e.g., CBP501) as set forth herein treatment.

In some embodiments a sample comprises a biological sample. In certain aspects, a sample can comprise a cell, tissue or organ (e.g., lung) biopsy, or a blood or serum sample.

In certain aspects a subject is a mammal. The mammal can be a human. In some aspects a subject is a human.

In various embodiments, expression is measured by a quantitative assay. In some aspects expression is measured with, or detected by contact with an analyte that detects an NRF2 protein or a protein encoded by an NRF2 target gene, or detects KEAP 1 protein or target gene, or a mutation that reduces or decreases activity, function or expression of KEAP 1. In some embodiments expression is measured or detected by contact with an analyte that detects an NRF2 transcript, or the transcript of an NRF2 target gene, or by sequencing a nucleic acid that comprises KEAP 1, or a mutation that reduces or decreases activity, function or expression of KEAP 1. In some embodiments expression is measured or detection is by an immunoassay. In some embodiments expression is measured or detection is by an antibody immunoassay. In some embodiments, expression is measured or detection is by a Western blot, ELISA, Northern blot, immunohistochemistry or immunocyotchemistry. In certain embodiments expression is measured or detection is by determining cDNA of NRF2 or cDNA of NRF2 target gene. In some aspects, expression is measured or detection is by reverse transcription of NRF2 RNA or NRF2 target gene RNA and polymerase chain reaction (RT-PCR) of NRF2 cDNA or NRF2 target gene cDNA, or reverse transcription of KEAP 1 RNA or KEAP 1 gene and polymerase chain reaction (RT-PCR) of KEAP 1 cDNA.

In certain embodiments, a peptide or peptidomimetic (e.g., CBP501) comprises a salt or a pro-drug thereof, or a salt of a pro-drug thereof. In various aspects a salt comprises a sodium, calcium, magnesium, nitrate, potassium, phosphate, sulfonate, fumarate, citrate, carbonate, ascorbate, succinate, trifluoroacetate or acetate salt.

In some embodiments, a method described herein comprises administering a G2 checkpoint inhibitor agent (e.g., a peptide or peptidomimetic such as CBP501). In some embodiments, a method described herein comprises administering a calmodulin binding agent.

In some embodiments, a method described herein includes administering a nucleic acid damaging agent to the subject (e.g., a molecule that binds to or intercalates in DNA).

In some embodiments, a method described herein comprises administering a peptide or peptidomimetic (e.g., CBP501), a G2 checkpoint inhibitor agent, a calmodulin binding agent or a nucleic acid damaging agent to the subject 1, 2, 3, 4, 5, or more times, alone, or in any combination.

In certain aspects a cancer comprises a lung cancer (NSCLC).

Moreover disclosed herein are a population of cancer cells, wherein the cells are fixed or immobilized on a substrate and the cells have bound thereto a reagent that detects NRF2 protein, or a protein encoded by an NRF2 target gene, or KEAP 1 protein or gene. In some embodiments, a population of cancer cells obtained from a subject are fixed or immobilized on a substrate and the cells have bound thereto a reagent that detects NRF2 protein, or a protein encoded by an NRF2 target gene, or KEAP 1 protein or gene. In certain aspects, the reagent that detects an NRF2 protein or a protein encoded by an NRF2 target gene or KEAP 1 protein comprises an antibody.

Still further disclosed herein are a population of cancer cells, wherein the cells are fixed or immobilized on a substrate and the cells have bound thereto a reagent that selectively binds to NRF2 transcript, or a transcript of an NRF2 target gene, or KEAP 1 transcript. In some embodiments, a population of cancer cells obtained from a subject are fixed or immobilized on a substrate and the cells have bound thereto a reagent that selectively binds to an NRF2 transcript, or a transcript of an NRF2 target gene, or KEAP 1 transcript. In certain aspects, the reagent that selectively binds to NRF2 transcript, or transcript of an NRF2 target gene comprises an oligonucleotide or primer.

NRF2 target genes include any of Glutathione reductase (GSR), Glucose-6-phosphate dehydrogenase (G6PD), ATP-binding cassette sub-familyC member2 (ABCC2), Aldo-keto reductase family1C1 (AKR1C1), Aldo-keto reductase family1C3 (AKR1C3), NAD(P)H dehydrogenase, quinonel (NQO1), AKR1B10, γ-glutamyl cysteine synthetase modifier subunit (γGCSm) or Glutathione peroxidasel (GPX1). In some aspects, a substrate comprises a glass or plastic plate, slide or chip.

Still further disclosed herein are compositions comprising nucleic acid isolated, purified or extracted from a population of cancer cells, where the nucleic acid comprises NRF2 gene or transcript thereof bound to a reagent that selectively binds to NRF2 gene or a transcript thereof, or binds to KEAP 1 gene or a transcript thereof. Compositions include nucleic acid isolated, purified or extracted from a population of cancer cells, wherein the nucleic acid comprises an NRF2 target gene or transcript thereof bound to a reagent that selectively binds to NRF2 gene or transcript thereof, or binds to KEAP 1 gene or a transcript thereof. In some aspects, a reagent that selectively binds to NRF2 transcript, or transcript of an NRF2 target gene, comprises an oligonucleotide or primer.

Yet further disclosed herein are compositions comprising protein isolated, purified or extracted from a population of cancer cells, where the protein comprises NRF2 protein bound to a reagent that selectively detects NRF2 protein, or KEAP 1 protein bound to a reagent that selectively detects KEAP 1 protein. Compositions include protein isolated, purified or extracted from a population of cancer cells, wherein the protein comprises a protein encoded by an NRF2 target gene bound to a reagent that selectively detects the protein encoded by an NRF2 target gene. In some aspects, a reagent that selectively detects NRF2 protein, or protein encoded by an NRF2 target gene, or KEAP 1 protein comprises an antibody.

NRF2 target genes include any of Glutathione reductase (GSR), Glucose-6-phosphate dehydrogenase (G6PD), ATP-binding cassette sub-familyC member2 (ABCC2), Aldo-keto reductase family1C1 (AKR1C1), Aldo-keto reductase family1C3 (AKR1C3), NAD(P)H dehydrogenase, quinonel (NQO1), AKR1B10, γ-glutamyl cysteine synthetase modifier subunit (γGCSm) or Glutathione peroxidasel (GPX1). In some aspects, a substrate comprises a glass or plastic plate, slide or chip.

### DESCRIPTION OF THE DRAWINGS

FIGS. 1A-1H show CBP501 sensitivity is characterized in both H1703 and H1437 NSCLC cell lines by lh co-treatment with CDDP.

FIGS. 1A-1D show H1703.

FIGS. 1E-1H show H1437. (FIGS. 1A, 1E) Cell cycle distribution change on subG1 population by flow-cytometry analysis (n=2). (FIGS. 1B, 1F) G1 phase (n=2). (FIGS. 1C, 1G) S phase (n=2). (FIGS. 1D, 1H) G2-M phase (n=2). Chi-square tests were performed to compare the change in the cell cycle distribution between CBP501 minus and plus at each CDDP dose point. An asterisk indicates statistical significance (p<0.001). N.S.=not significant (p>0.05). Error bars indicate the standard deviation from duplicate flowcytometry analyses.

FIGS. 2A-2D show microarray gene expression analyses indicating an increase in Nrf2-dependent gene expression in CBP501 insensitive cell lines.

FIG. 2A shows Heat-map display of the expression of isolated genes as determined by microarray analysis. Left panel comprises the CBP501 insensitive cell lines. Right panel comprises the CBP501 sensitive cell lines. Green indicates a low level of expression. Red indicates a high level of expression. Black indicates intermediate levels of expression (a value approx. 5000). Experiments were performed in triplicate.

FIG. 2B shows Western blot analysis for Nrf2 signaling components in NSCLCs.

FIG. 2C shows Quantitative analysis of Nrf2 protein levels in NSCLCs (n=3).

FIG. 2D shows Western blot analysis for a series of genes initially identified by microarray analysis.

FIGS. 3A-3C show a high degree of Nrf2 nuclear localization correlates with high levels of Nrf2 expression.

FIG. 3A shows Immunocytochemistry for Nrf2 in NSCLCs. Left panels show Nrf2 in green. Middle panels show Hoechst dye in red. Right panels show the merged images. Scale bar indicates 100 μm.

FIGS. 3B, 3C show Quantification of Nrf2 intensities from the five different images from FIG. 3A (n=5). (FIG. 3B) Nrf2 intensities in whole cells. (FIG. 3C) Nrf2 intensities in nuclei. Error bars signify standard deviations of Nrf2 intensity as determined from the five different images.

FIG. 4A-4D show Nrf2 activating agent Sulforaphane (SFN) cancels the effect of CBP501; knock-down of Nrf2 attenuates SFN's effect.

FIG. 4A shows Western blot analysis for a series of Nrf2 target proteins in NSCLCs.

FIG. 4B shows Confirmation of the efficiency of knock-down by Nrf2 shRNA lentivirus transfection. Upper bands indicated by black arrow head are Nrf2 protein levels. Middle and lower bands indicate internal controls of IQGAP1 and ATM respectively.

FIGS. 4C, 4D show Analysis of cell cycle distribution changes by using Flow-cytometry (n=4). SFN was subjected 24h to H1703 Control (Ctrl)-sh and Nrf2-sh sub-lines prior to lh treatment of CDDP (2.5 μg/m1)/CBP501 (1 μM). (FIG. 4C) Whole cell cycle distribution. (FIG. 4D) G2-M phase. An asterisk indicates statistical significance (TTEST; p<0.005). Error bars signify standard deviations as determined from four flowcytometric analyses.

FIGS. 5A-5C show knock-down of Nrf2 in the CBP501-insensitive cell line H1437, increases CBP501 sensitivity.

FIG. 5A shows Confirmation of knock-down efficiency by Nrf2 shRNA lentivirus infection. Upper bands indicate Nrf2 protein level. Middle three bands are the Nrf2 target molecules AKR1C3, G6PD and GSR respectively. Lower two bands indicate internal controls of IQGAP1 and MLC2 alpha respectively.

FIG. 5B shows One hour treatment with CDDP in combination with or without CBP501. Cell cycle distribution changes are detected by Flow-cytometry (n=4). The graphs at the left are from the sub-line established by introducing Control (Ctrl)-shRNA into the H1437. The graphs at the right are from the sub-line established by indtroducing Nrf2-shRNA into H1437. Upper graphs indicate the subGl population. Lower graphs indicate the G2-M population. An asterisk indicates statistical significance (TTEST; p<0.001). N.S.=not significant (p>0.01).

FIG. 5C showsGraphs of the difference analysis for the data from FIG. 5B. Blue bars indicate Control (Ctrl)-shRNA with CBP501 minus the cell line without CBP501. Red bars indicate Nrf2-sh with CBP501 minus the cell line without CBP501. Graph (left) indicates the percentage of cells in subG1. Graph (right) indicates the percentage in G2-M. An asterisk indicates statistical significance (TTEST; p<0.001) between red and blue bars. Error bars indicate the standard deviation found from four flowcytometry analyses.

FIGS. 6A-6E show Nrf2 target genes are good candidates as markers for predicting CBP501 sensitivity using their protein or mRNA expression levels.

FIG. 6A shows Heat-map display of isolated gene expression levels, as determined by microarray analysis, in a total of twenty-eight NSCLC cell lines. Left panel comprises the CBP501 insensitive cell lines. Right panel comprises the CBP501 sensitive cell lines. Green indicates a value indicating a low level of expression. Red indicates a high level of expression. Black indicates intermediate levels of expression (numerical value approx. 5000). Experiments performed in triplicate. Percentage of correlation between CBP501 sensitivity and isolated gene expression values are indicated at the right side of the heat-map panel.

FIG. 6B shows Western blot analysis for a series of Nrf2 target proteins in NSCLC cell lines extended beyond the original set (Table 2).

FIG. 6C shows Quantitative analysis of Nrf2 and AKR1C3 protein levels in NSCLCs from table1 and 2 (n=3).

FIG. 6D shows Immunocytochemistry for AKR1C3 in NSCLCs. Upper panels show AKR1C3 in green. Middle panels show Hoechst dye in red. Lower panels show the merged image. Scale bar indicates 100 μm.

FIG. 6E shows Quantification of the AKR1C3 intensities from C (n=5).

FIG. 7 shows comparative analysis of individual gene expression as in CBP501 insensitive cell lines or the CBP501 sensitive cell lines from FIG. 2. Black bars indicate the average values of expression in CBP501 insensitive cell lines. Red bars indicate the average values of expression in CBP501 sensitive cell lines.

FIGS. 8A-8B show immunocytochemistry for AKR1C1 in NSCLCs.

FIG. 8A shows Left panels show AKR1C1 in a pseudo-color of green. Middle panels show Hoescht in a pseudo-color of red. Right panels show the merged images. Scale bar indicates 100 microns.

FIG. 8B shows quatification of AKR1C1 intensities from FIG. 8A (n=5).

FIGS. 9A-9B show immunocytochemistry for AKR1B10 in NSCLCs.

FIG. 9A shows Left panels show AKR1B 10 in a pseudo-color of green. Middle panels show Hoescht in a pseudo-color of red. Right panels show the merged images. Scale bar indicates 100 microns.

FIG. 9B shows quatification of AKR1B10 intensities from FIG. 9A (n=5).

### DETAILED DESCRIPTION

In some embodiments, there are provided peptides and peptidomimetics employed in connection with the methods disclosed herein. In one embodiment, a peptide or peptidomimetic sequence includes the following structure: P1, P2, P3, P4, P5, P6 or P6, P5, P4, P3, P2, P1. P1 is Cha, Nal (2), (Phe-2,3,4,5,6-F), (Phe-3,4,5F), (Phe-4CF₃), an amino acid that occupies a similar side chain space (e.g., Tyr or Phe), or any amino acid with one or two aromatic, piperidine, pyrazine, pyrimidine, piperazine, morpholine or pyrimidine group(s), or one indole, pentalene, indene, naphthalene, benzofuran, benzothiophene, quinoline, indoline, chroman, quinoxaline, quinazoline group in the side chain; P2 is Cha, Nal(2), (Phe-2,3,4,5,6-F), (Phe-3,4,5F), (Phe-4CF₃), Bpa, Phe4NO2, an amino acid that occupies a similar side chain space (e.g., Tyr or Phe), or any amino acid with one or two aromatic, piperidine, pyrazine, pyrimidine, piperazine, morpholine or pyrimidine group(s), or one indole, pentalene, indene, naphthalene, benzofuran, benzothiophene, quinoline, indoline, chroman, quinoxaline, or quinazoline group in the side chain; P3, P4, P5 are any amino acid (e.g., P4 is Tip), or wherein one or more of P3, P4, P5 is a simple carbon chain (e g , 11-aminoundecanoic acid, 10-aminodecanoic acid, 9-aminononanoic acid, 8-aminocaprylic acid, 7-aminoheptanoic acid, 6-aminocaproic acid, or a similar structure with one or more unsaturated carbon bonds) such that the distance between P2 and P6 is about the same as the distance when each of P3, P4, P5 are amino acids; and P6 is Bpa, Phe4NO2, any one amino acid and Tyr (e.g., Ser-Tyr), any one amino acid and Phe (e.g., Ser-Phe), any amino acid, or nothing

In another embodiment, a peptide or peptidomimetic sequence includes the following structure: P1, P2, P3, P4, P5, P6; P6, P5, P4, P3, P2, P1; P1, P2, P3, P4, P5, P6, P7, P8, P9, P10, P11, P12; P1, P2, P3, P4, P5, P6, P12, P11, P10, P9, P8, P7; P6, P5, P4, P3, P2, P1, P7, P8, P9, P10, P11, P12; P6, P5, P4, P3, P2, Pl, P12, P11, P10, P9, P8, P7; P7, P8, P9, P10, P11, P12, P1, P2, P3, P4, P5, P6; P7, P8, P9, P10, P11, P12, P6, P5, P4, P3, P2, P1; P12, P11, P10, P9, P8, P7, P1, P2, P3, P4, P5, P6; P12, P11, P10, P9, P8, P7, P6, P5, P4, P3, P2, P1; P12, P1, P6, P9, P8, P7, P2, P1; P12, P11, P10, P6, P9, P4, P7, P2, P1; P1, P2, P7, P8, P9, P6, P11, P12; or P1, P2, P7, P4, P9, P6, P10, P11, P12. P1 is Cha, Nal(2), (Phe-2,3,4,5,6-F), (Phe-3,4,5F), (Phe-4CF3), Bpa, Phe4NO2, an amino acid that occupies a similar side chain space (e.g. d- or 1-Tyr, d- or 1-Phe), or any amino acid with one or two aromatic, piperidine, pyrazine, pyrimidine, piperazine, morpholine or pyrimidine group(s), or one indole, pentalene, indene, naphthalene, benzofuran, benzothiophene, quinoline, indoline, chroman, quinoxaline, or quinazoline group in the side chain; P2 is Cha, Nal(2), (Phe-2,3,4,5,6-F), (Phe-3,4,5F), (Phe-4CF3), or an amino acid that occupies a similar side chain space (e.g., Tyr or Phe), or any amino acid with one or two aromatic, piperidine, pyrazine, pyrimidine, piperazine, morpholine or pyrimidine group(s), or one indole, pentalene, indene, naphthalene, benzofuran, benzothiophene, quinoline, indoline, chroman, quinoxaline, quinazoline group in the side chain; P3, P4, P5 is any amino acid (e.g., P4 is Trp), or one or more of P3, P4, P5 is a simple carbon chain (e.g., 11-aminoundecanoic acid, 10-aminodecanoic acid, 9-aminononanoic acid, 8-aminocaprylic acid, 7-aminoheptanoic acid, 6-aminocaproic acid, or a similar structure with one or more unsaturated carbon bonds) such that the distance between P2 and P6 is about the same as the distance when each of P3, P4, P5 are amino acids; P6 is Bpa, Phe4NO2, any one amino acid and Tyr (e.g., Ser-Tyr), any one amino acid and Phe (e.g., Ser-Phe); and at least three of P7, P8, P9, P10, P11, P12 are basic amino acids with the rest being any amino acid or absent.

In a further embodiment, a peptide or peptidomimetic sequence includes the following structure: P1, P2, P3, P4, P5, P6, P7, P8, P9, P10, P11, P12; P12, P11, P10, P9, P8, P7, P6, P5, P4, P3, P2, P1; P12, P11, P10, P6, P9, P4, P7, P2, P1; or P1, P2, P7, P4, P9, P6, P10, P11, P12. P1 is Cha, Nal(2), (Phe-2,3,4,5,6-F), (Phe-3,4,5F), (Phe-4CF₃), Bpa, Phe4NO₂, an amino acid that occupies a similar side chain space, or any amino acid with one or two aromatic, piperidine, pyrazine, pyrimidine, piperazine, morpholine or pyrimidine group(s), or one indole, pentalene, indene, naphthalene, benzofuran, benzothiophene, quinoline, indoline, chroman, quinoxaline, or quinazoline group in the side chain; P2 is Cha, Nal(2), (Phe-2,3,4,5,6-F), (Phe-3,4,5F), (Phe-4CF3), an amino acid that occupies a similar side chain space (e.g., Tyr or Phe), or any amino acid with one or two aromatic, piperidine, pyrazine, pyrimidine, piperazine, morpholine or pyrimidine group(s), or one indole, pentalene, indene, naphthalene, benzofuran, benzothiophene, quinoline, indoline, chroman, quinoxaline, quinazoline group in the side chain; P3, P4, P5 are any amino acid (e.g., P4 is Tip), or one or more of P3, P4, P5 is a simple carbon chain (e g , aminoundecanoic acid or 8-aminocaprylic acid) such that the distance between P2 and P6 is about the same as the distance when each of P3, P4, P5 are amino acids; P6 is Bpa, Phe4NO2, any one amino acid and Tyr (e.g., Ser-Tyr), any one amino acid and Phe (e.g., Ser-Phe), any amino acid, or nothing; and at least three of P7, P8, P9, P10, P11, P12 are basic amino acids with the rest being any amino acid or absent.

In an additional embodiment, a peptide or peptidomimetic sequence includes the following structure: P1, P2, P3, P4, P5, P6 or P6, P5, P4, P3, P2, P1. P1 is Cha, Nal(2), (Phe-2,3,4,5,6-F), (Phe-3,4,5F), (Phe-4CF3), Bpa, Phe4NO2, Tyr, or Phe; P2 is Cha, Nal(2), (Phe-2,3,4,5,6-F), (Phe-3,4,5F), (Phe-4CF3), Bpa, Phe4NO2, Tyr, or Phe; P3 is Ser, Arg, Cys, Pro, or Asn; P4 is Trp; P5 is Ser, Arg, or Asn; or P3, P4, P5 is a single aminoundecanoic acid or a single 8-aminocaprylic acid; and P6 is Bpa, Phe4NO2, (Ser-Tyr), or (Ser-Phe).

In yet another embodiment, a peptide or peptidomimetic sequence includes the following structure: P1, P2, P3, P4, P5, P6, P7, P8, P9, P10, P11, P12; P1, P2, P3, P4, P5, P6, P12, P11, P10, P9, P8, P7; P6, P5, P4, P3, P2, P1, P7, P8, P9, P10, P11, P12; P6, P5, P4, P3, P2, P1, P12, P11, P10, P9, P8, P7; P7, P8, P9, P10, P11, P12, P1, P2, P3, P4, P5, P6; P7, P8, P9, P10, P11, P12, P6, P5, P4, P3, P2, P1; P12, P11, P10, P9, P8, P7, P1, P2, P3, P4, P5, P6; P12, P11, P10, P9, P8, P7, P6, P5, P4, P3, P2, P1; P12, P11, P6, P9, P8, P7, P2, P1; P12, P11, P10, P6, P9, P4, P7, P2, P1; P1, P2, P7, P8, P9, P6, P11, P12; or P1, P2, P7, P4, P9, P6, P10, P11, P12. P1 is Cha, Nal(2), (Phe-2,3,4,5,6-F), (Phe-3,4,5F), (Phe-4CF₃), Bpa, Phe4NO₂, Tyr, or Phe; P2 is Cha, Nal(2), (Phe-2,3,4,5,6-F), (Phe-3,4,5F), (Phe-4CF3), Bpa, Phe4NO2, Tyr, or Phe; P3 is Ser, Arg, Cys, Pro, or Asn; P4 is Trp; P5 is Ser. Arg, or Asn; or P3, P4, P5 is a single aminoundecanoic acid or a single 8-aminocaprylic acid; P6 is Bpa, Phe4NO₂, (d-Ser-d-Tyr), or (d-Ser-d-Phe); and at least three of P7, P8, P9, P10, P11, P12 are Arg or Lys with the rest being any amino acid or absent.

In still another embodiment, a peptide or peptidomimetic sequence includes the following structure: P1, P2, P3, P4, P5, P6, P7, P8, P9, P10, P11, P12; P12, P11, P10, P9, P8, P7, P6, P5, P4, P3, P2, P1; P12, P11, P10, P6, P9, P4, P7, P2, P1; or P1, P2, P7, P4, P9, P6, P10, P11, P12. P1 is Cha, or Nal(2); P2 is (Phe-2,3,4,5,6-F), (Phe-3,4,5F), (Phe-4CF₃); P3 is Ser; P4 is Trp; P5 is Ser or Asn; P6 is Bpa, Phe4NO₂, (Ser-Tyr), or (Ser-Phe); and at least three of P7, P8, P9, P10, P11, P12 are Arg with the rest being any amino acid or absent.

In yet an additional embodiment, a peptide or peptidomimetic sequence includes the following structure: P1, P2, P3, P4, P5, P6 or P6, P5, P4, P3, P2, P1. P1 is Cha, or Nal(2); P2 is (Phe-2,3,4,5,6-F), (Phe-3,4,5F) or (Phe-4CF₃); P3 is Ser; P4 is Trp; P5 is Ser; and P6 is Bpa, or (Ser-Tyr).

In yet a further embodiment, a peptide or peptidomimetic sequence comprising the following structure: P1, P2, P3, P4, P5, P6; P6, P5, P4, P3, P2, P1; P1, P2, P3, P4, P5, P6, P7, P8, P9, P10, P11, P12; P1, P2, P3, P4, P5, P6, P12, P11, P10, P9, P8, P7; P6, P5, P4, P3, P2, P1, P7, P8, P9, P10, P11, P12; P6, P5, P4, P3, P2, P1, P12, P11, P10, P9, P8, P7; P7, P8, P9, P10, P11, P12, P1, P2, P3, P4, P5, P6; P7, P8, P9, P10, P11, P12, P6, P5, P4, P3, P2, P1; P12, P11, P10, P9, P8, P7, P1, P2, P3, P4, P5, P6; P12, P11, P10, P9, P8, P7, P6, P5, P4, P3, P2, P1, P12, P11, P6, P9, P8, P7, P2, P1; P12, P11, P10, P6, P9, P4, P7, P2, P1; P1, P2, P7, P8, P9, P6, P11, P12 or P1, P2, P7, P4, P9, P6, P10, P11, P12. P1 is Cha, or Nal(2); P2 is (Phe-2,3,4,5,6-F), (Phe-3,4,5F) or (Phe-4CF₃); P3 is any amino acid (e.g., Ser, or Pro); P4 is d- or 1-Trp; P5 is any amino acid (e.g., Ser, or Pro); P6 is Bpa or (Ser-Tyr); P7 is Arg; P8 is Arg; P9 is Arg; P10 is Gln or Arg; P11 is Arg; and P12 is d- or 1-Arg.

In still further embodiments, a peptide or peptidomimetic sequence includes the following structure: P1, P2, P3, P4, P5, P6, P7, P8, P9, P10, P11, P12; P12, P11, P10, P9, P8, P7, P6, P5, P4, P3, P2, P1; P12, P11, P10, P6, P9, P4, P7, P2, P1; or P1, P2, P7, P4, P9, P6, P10, P11, P12. P1 is Cha or Nal(2); P2 is (Phe-2,3,4,5,6-F); P3 is Ser; P4 is Trp; P5 is Ser; P6 is Bpa or (Ser-Tyr); P7 is Arg; P8 is Arg; P9 is Arg; P10 is Gln or Arg; P11 is Arg; and P12 is Arg.

In particular aspects, a peptide or peptidomimetic sequence includes the following structure: (d-Bpa) (d-Ser)(d-Trp)(d-Ser) (d-Phe-2,3,4,5,6-F)(d-Cha)(d-Arg) (d-Arg) (d-Arg) (d-Gln)(d-Arg) (d-Arg) (CBP501; (d-Arg) (d-Arg) (d-Arg) (d-Gln)(d-Arg) (d-Arg) (d-Bpa)(d-Ser)(d-Trp)(d-Ser) (d-Phe-2,3,4,5,6-F) (d-Cha); (d-Bpa) (d-Ser)(d-Trp)(d-Ser) (d-Phe-2,3,4,5,6-F)(d-Cha)(d-Arg) (d-Arg) (d-Gln) (d-Arg) (d-Arg) (d-Arg); (d-Arg) (d-Arg) (d-Gln) (d-Arg) (d-Arg) (d-Arg) (d-Bpa) (d-Ser)(d-Trp)(d-Ser) (d-Phe-2,3,4,5,6-F) (d-Cha); (d-Cha) (d-Phe-2,3,4,5,6-F)(d-Ser)(d-Trp) (d-Ser) (d-Bpa) (d-Arg) (d-Arg) (d-Arg) (d-Gln)(d-Arg) (d-Arg) (d-Arg) (d-Arg) (d-Arg) (d-Gln)(d-Arg) (d-Arg) (d-Cha) (d-Phe-2,3,4,5,6-F)(d-Ser) (d-Trp)(d-Ser)(d-Bpa) (d-Cha)(d-Phe-2,3,4,5,6-F) (d-Ser)(d-Trp) (d-Ser) (d-Bpa) (d-Arg) (d-Arg) (d-Gln) (d-Arg) (d-Arg) (d-Arg); (d-Arg) (d-Arg) (d-Gln) (d-Arg) (d-Arg) (d-Arg) (d-Cha) (d-Phe-2,3,4,5,6-F) (d-Ser)(d-Trp) (d-Ser) (d-Bpa); (d-Arg) (d-Arg) (d-Arg) (d-Arg) (d-Arg) (d-Arg) (d-Cha) (d-Phe-2,3,4,5,6-F) (d-Ser) (d-Trp)(d-Ser) (d-Bpa); (d-Cha) (d-Phe-2,3,4,5,6-F) (d-Ser)(d-Trp)(d-Ser) (d-Bpa) (d-Arg)(d-Arg) (d-Arg)(d-Arg) (d-Arg) (d-Arg); (d-Arg) (d-Arg) (d-Arg) (d-Arg) (d-Arg) (d-Arg) (d-Bpa) (d-Ser)(d-Trp)(d-Ser) (d-Phe-2,3,4,5,6-F)(d-Cha); (d-Bpa) (d-Ser) (d-Trp)(d-Ser) (d-Phe-2,3,4,5,6-F)(d-Cha) (d-Arg) (d-Arg) (d-Arg) (d-Arg) (d-Arg) (d-Arg); (d-Arg)(d-Arg)(d-Bpa)(d-Arg)(d-Arg)(d-Arg) (d-Phe-2,3,4,5,6-F) (d-Cha); (d-Cha) (d-Phe-2,3,4,5,6-F) (d-Arg) (d-Arg) (d-Arg) (d-Bpa) (d-Arg)(d-Arg); (d-Arg) (d-Arg) (d-Arg) (d-Bpa) (d-Arg)(d-Trp) (d-Arg) (d-Phe-2,3,4,5,6-F)(d-Cha); (d-Cha) (d-Phe-2,3,4,5,6-F) (d-Arg)(d-Trp) (d-Arg) (d-Bpa) (d-Arg) (d-Arg) (d-Arg); (d-Arg) (d-Arg) (d-Arg) (d-Arg) (d-Bpa) (d-Arg)(d-Trp) (d-Arg) (d-Phe-2,3,4,5,6-F)(d-Cha); (d-Cha) (d-Phe-2,3,4,5,6-F) (d-Arg)(d-Trp) (d-Arg) (d-Bpa) (d-Arg) (d-Arg) (d-Arg) (d-Arg); (d-Arg) (d-Arg) (d-Arg)(d-Bpa)(d-Arg)(d-Arg) (d-Arg) (d-Phe-2,3,4,5,6-F)(d-Cha); or (d-Cha) (d-Phe-2,3,4,5,6-F) (d-Arg) (d-Arg) (d-Arg) (d-Bpa) (d-Arg) (d-Arg) (d-Arg); (d-Bpa)(d-Ser)(d-Trp)(d-Ser)(d-Phe-2,3,4,5,6-F) (d-Cha)(d-Arg) (d-Arg)(d-Arg)(d-Gln)(d-Arg)(d-Arg).

In some embodiments, there are provided physiologically acceptable salts of CBP501, for example, a salt of an inorganic base, a salt of an organic base, a salt of an inorganic acid, a salt of an organic acid, a salt of a basic or acidic amino acid and the like. Such salts can be produced by a method known methods (e.g., acetate salt can be produced by a step of liquid chromatography using acetate-containing solvent).

Examples of salts with inorganic base include alkali metal salt such as sodium salt, potassium salt and the like, alkaline earth metal salt such as calcium salt, magnesium salt and the like, and aluminum salt, ammonium salt and the like.

Examples of salts with organic base include salts with trimethylamine, triethylamine, pyridine, picoline, ethanolamine, diethanolamine, triethanolamine, tromethamine[tris(hydroxymethyl)aminomethane], tert-butylamine, cyclohexylamine, benzylamine, dicyclohexylamine, N,N′-dibenzylethylenediamine and the like.

Examples of salts with inorganic acid include salts with hydrochloric acid, hydrobromic acid, nitric acid, sulfuric acid, phosphoric acid and the like.

Examples of salts with organic acid include salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like.

Examples of salts with basic amino acid include salts with arginine, lysine, ornithine and the like.

Examples of salts with acidic amino acid include salts with aspartic acid, glutamic acid and the like.

In some embodiments, there are provided salts of an organic acid such as acetic acid and the like. The number of acetic acid attached to a peptide or peptidomimetic as set forth herein (e.g., CBP501), in particular, can vary, including, for example 4 or 5 acetic acids per a peptide or peptidomimetic as set forth herein (e.g., CBP501). Alternatively, a mixture of a peptide or peptidomimetic (e.g., CBP501) acetate salts having different number of acetic acids attached thereto (for example, mixture of 4 acetate and 5 acetate etc.) may be used.

In some embodiments, there is provided an acetate salt of a peptide compound CBP501: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg). A method of producing an acetate salt of a peptide compound shown by CBP501 may comprise a step of performing liquid chromatography using an acetate-containing solvent.

In additional aspects, peptides and peptidomimetic sequences include one or more 1-type or d-type residues; a d-residue substituted with a 1-residue; or a 1-residue substituted with a d-residue.

Peptides and peptidomimetic sequences include one or more of the following activities: inhibits proliferation of a cell; abrogates cell cycle G2 checkpoint of a cell; stimulates apoptosis of a cell; stimulates catastrophe of a cell.

Peptides and peptidomimetic sequences include a sequence having a length from about 6 to about 12, 10 to about 20, 18 to about 25, 25 to about 100, 25 to about 200, or 50 to about 300 residues in length.

Further provided are compositions including the peptides and peptidomimetic sequences disclosed herein. In one embodiment, a composition includes a peptide or peptidomimetic sequence and a nucleic acid damaging agent. In another embodiment, a composition includes a peptide or peptidomimetic sequence and an anti-proliferative agent. In an additional embodiment, a composition includes a pharmaceutically acceptable carrier or excipient and a peptide or peptidomimetic sequence and optionally a nucleic acid damaging agent or an anti-proliferative agent.

In some embodiments, methods include administering a nucleic acid damaging agent, a nucleic acid damaging treatment, an anti-proliferative agent, or an anti-proliferative treatment to the subject. In particular aspects, the agent or treatment comprises a drug (e.g., a chemotherapeutic drug such as 5-fluorouracil (5-FU), rebeccamycin, adriamycin (ADR), bleomycin (Bleo), pepleomycin, a cisplatin derivative such as cisplatin (CDDP) carboplatin, picoplatin, nedaplatin, miriplatin, satraplatin, triplatin, lipoplatin, mitaplatin, oxaliplatin, or camptotecin (CPT)), radiation (e.g., UV radiation, IR radiation, or alpha-, beta- or gamma-radiation), a radioisotope (e.g., I131, I125, 90Y, 177Lu, 213Bi, or 211At), environmental shock (e.g., hyperthermia).

As used herein, the terms “peptide,” “polypeptide” and “protein” are used interchangeably and refer to two or more amino acids covalently linked by an amide bond or non-amide equivalent. The peptides can be of any length. For example, the peptides can have from about 5 to 100 or more residues, such as, 5 to 12, 12 to 15, 15 to 18, 18 to 25, 25 to 50, 50 to 75, 75 to 100, or more in length. The peptides may include l- and d-isomers, and combinations of l- and d-isomers. The peptides can include modifications typically associated with post-translational processing of proteins, for example, cyclization (e.g., disulfide or amide bond), phosphorylation, glycosylation, carboxylation, ubiquitination, myristylation, or lipidation.

Peptides disclosed herein further include compounds having amino acid structural and functional analogues, for example, peptidomimetics having synthetic or non-natural amino acids or amino acid analogues, so long as the mimetic has one or more functions or activities. The compounds disclosed herein therefore include “mimetic” and “peptidomimetic” forms.

As used herein, the terms “mimetic” and “peptidomimetic” refer to a synthetic chemical compound which has substantially the same structural and/or functional characteristics of the peptides. The mimetic can be entirely composed of synthetic, non-natural amino acid analogues, or can be a chimeric molecule including one or more natural peptide amino acids and one or more non-natural amino acid analogs. The mimetic can also incorporate any number of natural amino acid conservative substitutions as long as such substitutions do not destroy the mimetic's activity. As with polypeptides which are conservative variants, routine testing can be used to determine whether a mimetic has the requisite activity, e.g., that it has detectable cell cycle G2 checkpoint abrogating activity. A mimetic, when administered to a subject or contacted on a cell, that detectably disrupts the G2 cell cycle checkpoint, would therefore have G2 checkpoint abrogating activity.

Peptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., induce or stabilize a secondary structure, e.g., a beta turn, gamma turn, beta sheet, alpha helix conformation, and the like. For example, a polypeptide can be characterized as a mimetic when one or more of the residues are joined by chemical means other than an amide bond. Individual peptidomimetic residues can be joined by amide bonds, non-natural and non-amide chemical bonds other chemical bonds or coupling means including, for example, glutaraldehyde, N-hydroxysuccinimide esters, bifunctional maleimides, N,N′-dicyclohexylcarbodiimide (DCC) or N,N′-diisopropylcarbodiimide (DIC). Linking groups alternative to the amide bond include, for example, ketomethylene (e.g., —C(═O)—CH2- for −C(═O)—NH—), aminomethylene (CH2-NH), ethylene, olefin (CH═CH), ether (CH2-O), thioether (CH2-S), tetrazole (CN4-), thiazole, retroamide, thioamide, or ester (see, e.g., Spatola (1983) in Chemistry and Biochemistry of Amino Acids, Peptides and Proteins, Vol. 7, pp 267-357, “Peptide and Backbone Modifications,” Marcel Decker, N.Y.).

As discussed, a peptide can be characterized as a mimetic by containing one or more non-natural residues in place of a naturally occurring amino acid residue. Non-natural residues are known in the art. Particular non-limiting examples of non-natural residues useful as mimetics of natural amino acid residues are mimetics of aromatic amino acids include, for example, D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acid. Aromatic rings of a non-natural amino acid that can be used in place of a natural aromatic rings include, for example, thiazolyl, thiophenyl, pyrazolyl, benzimidazolyl, naphthyl, furanyl, pyrrolyl, and pyridyl aromatic rings.

Mimetics of acidic amino acids can be generated by substitution with non-carboxylate amino acids while maintaining a negative charge; (phosphono) alanine; and sulfated threonine. Carboxyl side groups (e.g., aspartyl or glutamyl) can also be selectively modified by reaction with carbodiimides (R′—N—C—N—R′) including, for example, 1-cyclohexyl-3(2-morpholinyl-(4-ethyl) carbodiimide or 1-ethyl-3(4-azonia-4,4-dimetholpentyl) carbodiimide Aspartyl or glutamyl groups can also be converted to asparaginyl and glutaminyl groups by reaction with ammonium ions.

Mimetics of basic amino acids can be generated by substitution, for example, in addition to lysine and arginine, with the amino acids ornithine, citrulline, or (guanidino)-acetic acid, or (guanidino)alkyl-acetic acid, where alkyl can be substituted or unsubstituted methyl, ethyl, propyl, hexyl, butyl, pentyl, isopropyl, iso-butyl, sec-isotyl, iso-pentyl, or a non-acidic amino acid. Nitrile derivative (e.g., containing the CN-moiety in place of COOH) can be substituted for asparagine or glutamine. Asparaginyl and glutaminyl residues can be deaminated to the corresponding aspartyl or glutamyl residues.

Arginine mimetics can be generated by reacting arginyl with one or more reagents including, for example, phenylglyoxal, 2,3-butanedione, 1,2-cyclohexanedione, or ninhydrin, optionally under alkaline conditions. Tyrosine residue mimetics can be generated by reacting tyrosyl with aromatic diazonium compounds or tetranitromethane. N-acetylimidizol and tetranitromethane can be used to form O-acetyl tyrosyl species and 3-nitro derivatives, respectively.

Lysine mimetics can be generated (and amino terminal residues can be altered) by reacting lysinyl with succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O -methytisourea, 2,4, pentanedione, and transamidase-catalyzed reactions with glyoxylate.

Methionine mimetics can be generated by reaction with methionine sulfoxide. Proline mimetics of include, for example, pipecolic acid, thiazolidine carboxylic acid, 3- or 4-hydroxy proline, dehydroproline, 3- or 4-methylproline, and 3,3,-dimethylproline. Histidine mimetics can be generated by reacting histidyl with diethylprocarbonate or para-bromophenacyl bromide. Other mimetics include, for example, those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

One or more residues can also be replaced by an amino acid (or peptidomimetic residue) of the opposite chirality. Thus, any amino acid naturally occurring in the L-configuration (which can also be referred to as R or S, depending upon the structure of the chemical entity) can be replaced with the same amino acid or a mimetic, but of the opposite chirality, referred to as the D-amino acid, but which can additionally be referred to as the R- or S-form.

Peptides and peptidomimetics further include modified forms of the sequences set forth herein, provided that the modified form retains, at least a part of, the function of the unmodified or reference peptide or peptidomimetic. For example, a modified peptide or peptidomimetic will retain at least a part of cell proliferative inhibiting or G2 abrogating activity, but may have increased or decreased cell proliferative inhibiting or G2 abrogating activity relative to reference peptide or peptidomimetic.

Modified peptides and peptidomimetics can have one or more amino acid residues substituted with another residue, added to the sequence or deleted from the sequence. In one embodiment, the modified peptide or peptidomimetic has one or more amino acid substitutions, additions or deletions (e.g., 1-3,3-5, 5-10 or more). In one aspect, the substitution is with an amino acid or mimetic whose side chain occupies a similar space with the reference amino acid or mimetic (the amino acid or mimetic that is being substituted). In still another aspect, the substitution is with a non-human amino acid which is structurally similar to the human residue. In a particular aspect, the substitution is a conservative amino acid substitution.

As used herein, the term “similar space” means a chemical moiety that occupies a three-dimensional space similar in size to a reference moiety. Typically, a moiety that occupies a similar space will be similar in size to the reference moiety. An amino acid or mimetic that “occupies a similar side chain space” has a side chain that occupies a three-dimensional space similar in size to the reference amino acid or mimetic. Specific examples for d-(Phe-2,3,4,5,6-F), 1-(Phe-2,3,4,5,6-F), d-(Phe-3,4,5F), 1-(Phe-3,4,5F), d-(Phe-4CF₃) or 1-(Phe-4CF₃), are (1 or d-Phe-2R1,3R2,4R3,5R4,6R5) where R1,R2,R3,R4,R5 can be chloride, bromide, fluoride, iodide, hydrogen, hydrogen oxide or absent. For small molecules, e.g., fluoride which has a size of about 1 Angstrom, similar space may be absence of a moiety.

The term “conservative substitution” means the replacement of one amino acid by a biologically, chemically or structurally similar residue. Biologically similar means that the substitution is compatible with biological activity, e.g., anti-cell proliferative or G2 abrogating activity. Structurally similar means that the amino acids have side chains with similar length, such as alanine, glycine and serine, or having similar size. Chemical similarity means that the residues have the same charge or are both hydrophilic or hydrophobic. Particular examples include the substitution of one hydrophobic residue, such as isoleucine, valine, leucine or methionine for another, or the substitution of one polar residue for another, such as the substitution of arginine for lysine, glutamic for aspartic acids, or glutamine for asparagine, serine for threonine, and the like.

Peptides and peptidomimetics therefore include peptides and peptidomimetics having a sequence that is not identical to a sequence of peptides and peptidomimetics sequences set forth in Table 1. In one embodiment, a peptide or peptidomimetic has a sequence having 50%, 60%, 70%, 75%, 80%, 85%, 90%, 95%, or more identity with a sequence set forth in Table 1. In one aspect, the identity is over a defined area of the sequence, e.g., the amino or carboxy terminal 3-5 residues.

The peptides and peptidomimetics can be produced and isolated using any method known in the art. Peptides can be synthesized, whole or in part, using chemical methods known in the art (see, e.g., Caruthers (1980) Nucleic Acids Res. Symp. Ser. 215-223; Horn (1980) Nucleic Acids Res. Symp. Ser. 225-232; and Banga, A. K., Therapeutic Peptides and Proteins, Formulation, Processing and Delivery Systems (1995) Technomic Publishing Co., Lancaster, Pa.). Peptide synthesis can be performed using various solid-phase techniques (see, e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the manufacturers instructions.

Individual synthetic residues and polypeptides incorporating mimetics can be synthesized using a variety of procedures and methodologies known in the art (see, e.g., Organic Syntheses Collective Volumes, Gilman, et al. (Eds) John Wiley & Sons, Inc., NY). Peptides and peptide mimetics can also be synthesized using combinatorial methodologies. Techniques for generating peptide and peptidomimetic libraries are well known, and include, for example, multipin, tea bag, and split-couple-mix techniques (ses, for example, al-Obeidi (1998) Mol. Biotechnol. 9:205-223; Hruby (1997) Cliff. Opin. Chem. Biol. 1:114-119; Ostergaard (1997) Mol. Divers. 3:17-27; and Ostresh(1996) Methods Enzymol. 267:220-234). Modified peptides can be further produced by chemical modification methods (see, for example, Belousov (1997) Nucleic Acids Res. 25:3440-3444; Frenkel (1995) Free Radic. Biol. Med. 19:373-380; and Blommers (1994) Biochemistry 33:7886-7896).

Peptides can also be synthesized and expressed as fusion proteins with one or more additional domains linked thereto for producing a more immunogenic peptide, to more readily isolate a recombinantly synthesized peptide, or to identify and isolate antibodies or antibody-expressing B cells. Domains facilitating detection and purification include, for example, metal chelating peptides such as polyhistidine tracts and histidine-tryptophan modules that allow purification on immobilized metals; protein A domains that allow purification on immobilized immunoglobulin; and the domain utilized in the FLAGS™ extension/affinity purification system (Immunex Corp, Seattle, Wash.) The inclusion of a cleavable linker sequence such as Factor Xa or enterokinase (Invitrogen, San Diego Calif.) between a purification domain and the peptide can be used to facilitate peptide purification. For example, an expression vector can include a peptide-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-14). The histidine residues facilitate detection and purification of the fusion prtein while the enterokinase cleavage site provides a means for purifying the peptide from the remainder of the fusion protein. Technology pertaining to vectors encoding fusion proteins and application of fusion proteins is known in the art (see e.g., Kroll (1993) DNA Cell. Biol., 12:441-53).

In some embodiments there are provided nucleic acids encoding the peptides disclosed herein. In particular embodiments, a nucleic acid encodes peptide sequences herein having a length of about 8 to 12, 12 to 15, 15 to 18, 15 to 20, 18 to 25, 20 to 25, 25 to 35, 25 to 50 or 50 to 100 amino acids or more in length.

The terms “nucleic acid” and “polynueleotide” are used interchangeably herein to refer to all forms of nucleic acid, including deoxyribonucleic acid (DNA) and ribonucleic acid (RNA). The nucleic acids can be double, single strand, or triplex, linear or circular Nucleic acids include genomic DNA, cDNA, and antisense. RNA nucleic acid can be spliced or unspliced mRNA, rRNA, tRNA or antisense (e.g., RNAi). Nucleic acids include naturally occurring, synthetic, as well as nucleotide analogues and derivatives. Such altered or modified polynucleotides include analogues that provide nuclease resistance, for example. Nucleic acid lengths also can be less than the exemplified peptide sequences. For example, a subsequence of any of the peptide sequences can encode a peptide having anti-proliferative or G2 abrogating activity.

Nucleic acid can be produced using any of a variety of well known standard cloning and chemical synthesis methods and can be altered intentionally by site-directed mutagenesis or other recombinant techniques known to those skilled in the art. Purity of polynucleotides can be determined through sequencing, gel electrophoresis and the like.

Nucleic acids may be inserted into a nucleic acid construct in which expression of the nucleic acid is influenced or regulated by an “expression control element,” the combination referred to as an “expression cassette.” The term “expression control element” means one or more sequence elements that regulates or influences expression of a nucleic acid sequence to which it is operatively linked. An expression control element operatively linked to a nucleic acid sequence controls transcription and, as appropriate, translation of the nucleic acid sequence.

The term “operatively linked” refers to a functional juxtaposition wherein the components so described are in a relationship permitting them to function in their intended manner. Typically expression control elements are juxtaposed at the 5′ or at the 3′ ends of the gene but can also be intronic. Promoters are generally positioned 5′ of the coding sequence. A “promoter” is meant a minimal sequence element sufficient to direct transcription.

Expression control elements include promoters, enhancers, transcription terminators, gene silencers, a start codon (e.g., ATG) in front of a protein-encoding gene. Expression control elements activate constitutive transcription, inducible transcription (i.e., require an external signal for activation), or derepress transcription (i.e., a signal turns transcription off; removing the signal activates transcription). Expression cassettes can also include control elements sufficient to render gene expression controllable for specific cell-types or tissues (i.e., tissue-specific control elements).

Nucleic acids may be inserted into a plasmid for propagation into a host cell and for subsequent genetic manipulation. A plasmid is a nucleic acid that can be stably propagated in a host cell; plasmids optionally contain expression control elements in order to drive expression of the nucleic acid encoding peptide in the host cell. The term “vector” is used herein synonymously with a plasmid and may also include an expression control element for expression in a host cell. Plasmids and vectors generally contain at least an origin of replication for propagation in a cell and a promoter. Plasmids and vectors are therefore useful for genetic manipulation of peptide encoding nucleic acids, for producing peptides, and for expressing the peptides in host cells or whole organisms, for example.

Peptides may therefore be expressed in bacterial systems using constitutive promoters such as T7, or inducible promoters such as pL of bacteriophage π, plac, ptrp, ptac (ptrp-lac hybrid promoter); in yeast systems using constitutive promoters such as ADH or LEU2 or an inducible promoter such as GAL (see, e.g., Ausubel et al., In: Current Protocols in Molecular Biology, Vol. 2, Ch. 13, ed., Greene Publish. Assoc. & Wiley Interscience, 1988; Grant et al. Methods in Enzymology, 153:516 (1987), eds. Wu & Grossman; Bitter Methods in Enzymology, 152:673 (1987), eds. Berger & Kimmel, Acad. Press, N.Y.; and, Strathern et al., The Molecular Biology of the Yeast Saccharomyces (1982) eds. Cold Spring Harbor Press, Vols. I and II; R. Rothstein In: DNA Cloning, A Practical Approach, Vol. 11, Ch. 3, ed. D. M. Glover, IRL Press, Wash., D. C., 1986); in insect cell systems using constitutive or inducible promoters such as ecdysone; and in mammalian cell systems using constitutive promoters such as SV40, RSV, or inducible promoters derived from the genome of mammalian cells such as metallothionein IIA promoter, heat shock promoter, or derived from mammalian virus such as adenovirus late promoter or the inducible mouse mammary tumor virus long terminal repeat. Peptide expression systems further include vectors designed for in vivo use including adenoviral vectors (U.S. Pat. Nos. 5,700,470 and 5,731,172), adeno-associated vectors (U.S. Pat. No. 5,604,090), herpes simplex virus vectors (U.S. Pat. No. 5,501,979) and retroviral vectors (U.S. Pat. Nos. 5,624,820, 5,693,508 and 5,674,703 and WIPO publications WO92/05266 and WO92/14829). Bovine papilloma virus (BPV) has also been employed in gene therapy (U.S. Pat. No. 5,719,054). Such gene therapy vectors also include CMV based vectors (U.S. Pat. No. 5,561,063).

### EXAMPLES

CBP501 is an anti-cancer drug candidate that was investigated in two randomized Phase II clinical trials for patients with non-squamous non-small cell lung cancer (NSCLC) and malignant pleural mesothelioma (MPM). CBP501 has been shown to have two mechanisms of action, namely calmodulin modulation and G2 checkpoint abrogation. A biomarker to predict sensitivity to CBP501 is disclosed herein. Twenty-eight NSCLC cell lines were classified into two subgroups, CBP501 sensitive and insensitive, by quantitatively analyzing the cis-diamminedichloro-platinum (II) (CDDP)-enhancing activity of CBP501 through treatments with short term (1 hour) co-exposure to CDDP and CBP501 or to either alone. Microarray analysis was performed on these cell lines to identify gene expression patterns that correlated with CBP501 sensitivity. It was found that multiple Nuclear factor erythroid-2 related factor2 (Nrf2) target genes showed high expression in CBP501-insensitive cell lines. Western blot and imunnocytochemical analysis for Nrf2 in NSCLC cell lines also indicated higher protein level in CBP501 insensitive cell lines. Moreover, CBP501 sensitivity is modulated by silencing or Sulforaphane (SEN) induced over-expression of Nrf2. These results indicate that Nrf2 transcriptional factor is a potential candidate as a biomarker for resistance to CBP501. This Example identifies those subpopulations of patients who would respond well to the CBP501 and CDDP combination treatment for NSCLC.

Establishing quantifiable markers that predict sensitivity to a particular drug is an aim of personalized medicine. Several anti-cancer drugs have been developed based on known oncogenic mutations and the presence of the mutation by itself has been found to be a useful biomarker for these drugs. Examples include some tyrosine kinase inhibitors, including gefitinib and erlotinib, that target epidermal growth factor receptor (EGFR) in NSCLC and for which efficacy depends on the presence of particular EGFR mutations (1, 2); Crizotinib, which is highly effective when NSCLC cells harbor the EML4-ALK fusion gene (3); and the BRAF inhibitor Vemurafenib, which is highly effective when melanoma cells carry the V600E mutation of BRAF (4).

It has been indicated that a combination of CDDP plus CBP501 showed evidence of clinical activity in patients with platinum-resistant ovarian carcinoma and MPM (5). The primary endpoint for efficacy was achieved on MPM (6). G2 checkpoint-abrogation had been proposed as a mechanism of action (MOA) based on the observation that CBP501 (i) inhibits multiple kinases that phosphorylate CDC25C at Ser216, (ii) binds directly to 14-3-3 protein, which forms suppressive complexes with phospho-CDC25C, (iii) attenuates phosphorylation of CDC25C at Ser216 and (iv) reduces the accumulation of cancer cells at G2/M upon lengthy combined exposure with CDDP or bleomycin (BLM) (7, 8).

An additional MOA for CBP501was demonstrated. This entails direct interaction of CBP501 with calmodulin, which leads to increased uptake of CDDP into cancer cells upon much shorter exposure to and at lower doses of CBP501 than the conditions required for G2 checkpoint abrogation (9). CDDP uptake is known to depend on multiple transporters and channels (10). Calmodulin-CBP501 interaction might affect a multiple transporter to increase CDDP uptake into cancer cells.

Nrf2 is a transcription factor that regulates the expression of many antioxidant genes, including those related to glutathione synthesis (11). In tumorigenesis, expression of particular oncogenic alleles of Kras, BRAF and Myc increases the levels of intracellular antioxidants by inducing Nrf2 expression (12). Nrf2 also regulates the expression of genes related to drug detoxification and transport (13).

This Example focuses on CBP501's effect on promoting CDDP uptake rather than G2 checkpoint abrogation by examining cells only upon short-term exposure to CBP501 and CDDP. Twenty-eight NSCLC cell lines were quantitatively defined as sensitive or insensitive to CBP501 by analyzing their response to a short (1h) co-exposure with CDDP and CBP501 as compared with either drug alone. Comprehensive gene expression analysis (microarray) was performed on these cell lines to identify the sensitivity markers for this MOA of CBP501. Multiple genes regulated by the Nrf2 transcription factor were identified as having high expression in CBP501 insensitive cell lines. Additionally, knock-down of Nrf2 by using Nrf2 sh-RNA revealed that Nrf2 expression is required for resistance to CBP501.

Materials and Methods

Cell culture and reagents. Human NSCLC cell lines were cultured in a variety of media, each supplemented with 10% fetal bovine serum (Invitrogen, San Diego, CA) at 37° C. with 5% CO₂/air. The media used was RPMI1640 (Sigma-Aldrich, St. Louis, Mo.) for NCI-H1755, NCI-H2030, NCI-H1437, NCI-H2122, NCI-H2172, NCI-H2228, NCI-H1563, NCI-H1944, NCI-H1993, NCI-H1734, NCI-H1568, NCI-H2444, NCI-H2291, NCI-H2347, NCI-H1838, NCI-H1299, HCC827, NCI-H1975, NCI-H1155, NCI-H522 and NCI-H1703; RPMI1640 supplemented with 2mM L-glutamine (Invitrogen) for NCI-H727; RPMI1640 supplemented with 4.5 g/L D-glucose (Sigma-Aldrich), 10 mM HEPES (Sigma-Aldrich) and 1 mM sodium pyruvate (Sigma-Aldrich) for NCI-H358, NCI-H520, NCI-H23, NCI-H647 and NCI-H838; Ham's F12K (Sigma-Aldrich) for A549. CDDP was purchased from Sigma-Aldrich. Sulforaphane (SFN) was purchased from Sigma-Aldrich. All NSCLC cell lines were purchased from ATCC and STR analysis had been performed by ATCC to authenticate the identity of each cell line. All cell lines were used within three months after thawing.

Cell cycle analysis. Cells were plated in 24-well plates and incubated for 24 hours. The cells were treated with or without CDDP plus or minus CBP501 at the indicated concentration for the indicated times. The cells were harvested and stained with Krishan's solution (0.1% sodium citrate, 50 μg/ml propidium iodide, 20 μg/ml RNase A, 0.5% NP-40). Stained cells were analyzed by FACSCalibur (Becton Dickinson, Franklin Lakes, N.J.) using CELLQuest software (Becton Dickinson).

Antibodies. Antibodies were purchased from the indicated sources: anti-NR0B1, anti-Grx, anti-Prx, anti-γGCSc, anti-γGCSm and anti-Gpxl (SantaCruz, Dallas, Tex.); anti-MLC, anti-G6PD, anti-ABCC2, anti-KEAP1, anti-BACH1, anti-Trxl, anti-SOD1, anti-HO1, anti-NQO1, anti-IQGAP1 and anti-ATM (Cell Signaling Technology, Beverly, Mass.); anti-GSR, anti-AKR1C1, anti-AKR1C3, anti-AKR1B10 and anti-Nrf2 (Epitomics, San Francisco, Calif.).

Microarray and Gene Expression Analysis. NSCLC cell lines were grown in a monolayer prior to RNA isolation. Total RNA was isolated using RNeasy Mini Kit (Qiagen, Tokyo, Japan). Agilent Expression Array analysis of the isolated total RNA samples was performed at TAKARABIO Inc. (Shiga, Japan). Each analysis of total RNA of a cell line was performed in triplicate. Preliminary statistical treatment (One-way ANOVA and Benj amini-Hochberg method: False Discovery Rate≦0.05) of the gene expression data was performed by TAKARABIO Inc.

Lentivirus infection. Nrf2 shRNA lentivirus particles and Control shRNA lentivirus particles were purchased from SantaCruz. Lentivirus infection was performed according to manufacturer's instructions. Cells (50% confluence) were treated with 5 μg/ml Polybrene (SantaCruz) prior to virus infection. One day after addition of virus, the cells were transferred to fresh medium and cells were selected by treatment with Puromycin (SantaCruz).

Immunofluorescence. Cells growing on coverslips were fixed with 4% paraformaldehyde (15 min), washed with phosphate-buffered saline (PBS) (five minutes, three times) and then blocked in 1×PBS with 1% Block Ace (DS pharma, Osaka, Japan). They were then incubated with antibodies using standard protocols. Primary antibodies included anti-Nrf2 (1:100), anti-AKR1C1(1:100), anti-AKR1C3(1:100) and anti-AKR1B10 (1:100). Secondary antibodies were labeled with Alexa-488 (Invitrogen) and Hoechst 33342 (DOJINDO, Kumamoto, Japan) was used to stain DNA. Confocal microscopy was performed using FV10i confocal microscope (OLYMPUS, Tokyo, Japan).

Western blot analysis. Cells (50% confluence) were treated with or without SFN at indicated concentrations for the indicated times. The cells were harvested and lysed (30 min on ice) in lysis buffer [50 mM Tris-HCl (pH 8.0), 5 mM EDTA (pH 8.0), 100 mM NaCl, 0.5% NP-40, 2 mM DTT, 50 mM NaF, 1 mM Na₃VO₄, 1 μM microcystin, proteinase inhibitors cocktail (Roche, Mannheim, Germany)]. The lysates were clarified by centrifugation (20600 g, 4° C.), and the supernatants were assayed for protein content using the detergent-compatible protein assay kit (Bio-Rad, Hercules, Calif.) according to the manufacturer's instructions. The whole cell lysates (60 μg) were run on 10-12% SDS-PAGE. Protein from each gel was transferred onto a polyvinylidene difluoride, (PVDF), membrane (Bio-Rad). The membrane was blocked at room temperature for 1 h in TBST (10 mM Tris-HCl [pH 8.0], 150 mM NaCl and 0.05% Tween-20) containing 1% Block Ace (DS pharma) and incubated with primary antibody overnight at 4° C. The membrane was incubated further with anti-peroxidase conjugated secondary antibody (Cell Signaling) for one hour at room temperature after washing, and the signals were detected using the enhanced chemiluminescence detection system (ECL Advance Western Blotting Detection Kit, GE Healthcare). Detected bands were quantified using Lumino-mater LAS-4000 instrument (Fujifilm, Tokyo, Japan).

Mutation search for NSCLC cell lines. Searching for Keapl mutations in NSCLC cell lines was performed using the on-line database, Catalogue of somatic mutations in cancer (COSMIC) (http://cancer.sanger.ac.uk/cancergenome/projects/cosmic/).

Results

NSCLC cell lines and the sensitivity to CBP501

CBP501 sensitizes tumor cells to CDDP, as shown by various methods including colony formation assays, in vitro viability assays, and flow-cytometry analysis, which measures systematic changes in cell cycle distribution. The results between the different methods correlate well (8, 9) and differential sensitivity to CBP501 varies consistently for different cell lines (9).

Here, the change in the cell cycle distribution, as indicated by flow-cytometry, was used to estimate the sensitivity of NSCLC cell lines to CBP501-enhanced cytotoxicity to CDDP. Such changes were monitored in cells treated with CDDP in the presence or absence of CBP501. The utility of this analysis for assessing the effectiveness of anti-cancer therapy is based on the absence of functioning G1/S checkpoint regulation in the cell cycle of many types of cancer cells (14). Lack of a functional G1/S checkpoint leads to an accumulation of cancer cells in G2/M phase upon their exposure to DNA-damaging anticancer agents (14, 15).

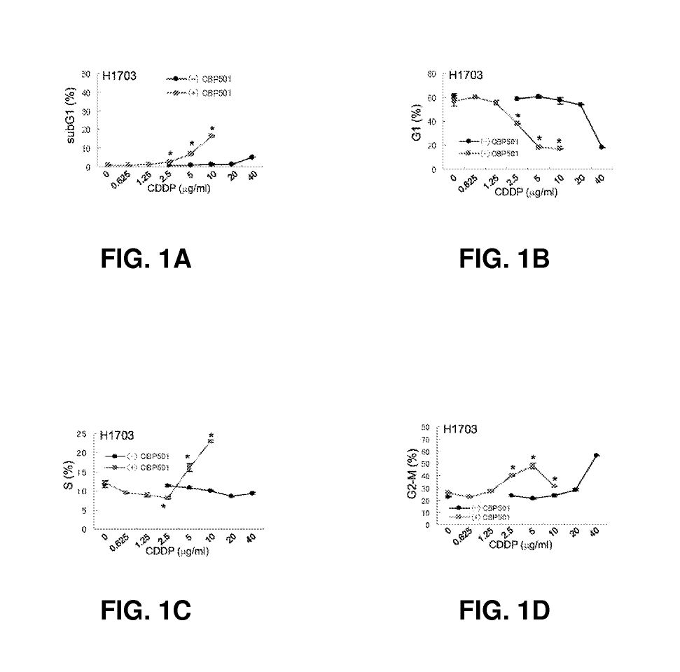

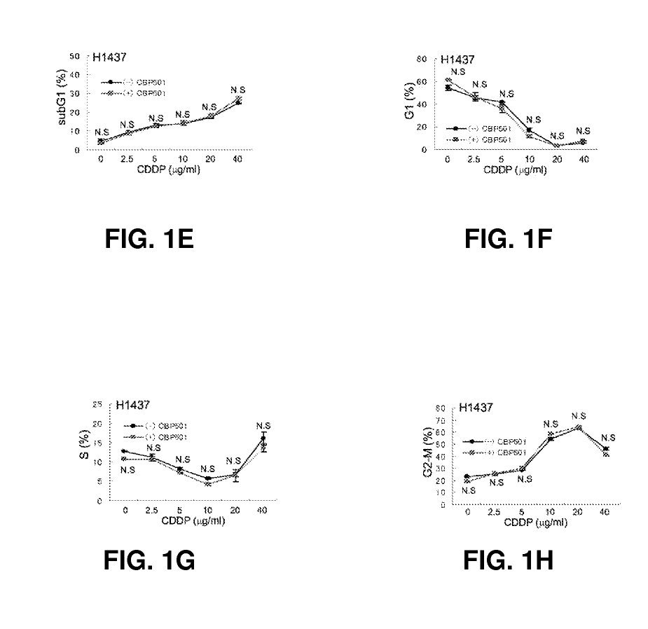

To assess the CDDP-enhancing activity of CBP501, one can examine either of two dose-response curves in which the X-axis indicates the dose of CDDP and Y-axis indicates either the number of cells in subG1 or in G2/M. The transition and peak in either dose-response curve will shift to the left in the presence of CBP501 for CBP501-sensitive cells, indicating the enhanced ability of CDDP to change the cell cycle distribution. By such an analysis of NCI-H1703 cells, the presence of CBP501 resulted in an approximate eight-fold increase in effectiveness of CDDP to alter the cell cycle distribution (FIG. 1A-D). On the other hand, the NCI-H1437 cell line did not exhibit any detectable shift in the dose-response curve of CDDP caused by CBP501 (FIG. 1E-H). Using this criterion based on CBP501's ability to shift the CDDP-induced subG1 or G2/M dose-response curve, twelve NSCLC cell lines were classified as being either, CBP501-sensitive or CBP501-insensitive. Cell lines that shifted the curve two fold or more were classified as CBP501 sensitive cells and those that shifted the curve less than two fold were classified as CBP501 insensitive (Table 1). Classification of CBP501 sensitivity in NSCLC cell lines.

TABLE 1

| | Folds increase of CDDP | Definition of CBP501 |
|---|---|---|
| Cell line's name | effect by CBP501 | sensitivity |
| NCI-H2030 | 1 | Insensitive |
| NCI-H1437 | 1 | Insensitive |
| NCI-H2122 | 1 | Insensitive |
| NCI-H2172 | 1.5 | Insensitive |
| NCI-H2228 | 1.5 | Insensitive |
| NCI-H1944 | 1.5 | Insensitive |
| NCI-H1568 | 2 | Sensitive |
| NCI-H2444 | 2 | Sensitive |
| NCI-H2291 | 2 | Sensitive |
| NCI-H2347 | 4 | Sensitive |
| NCI-H838 | 4 | Sensitive |
| NCI-H1703 | 8 | Sensitive |

Comprehensive gene expression analysis indicates that some Nrf2 target genes might have up-regulated expression in CBP501-insensitive cell lines

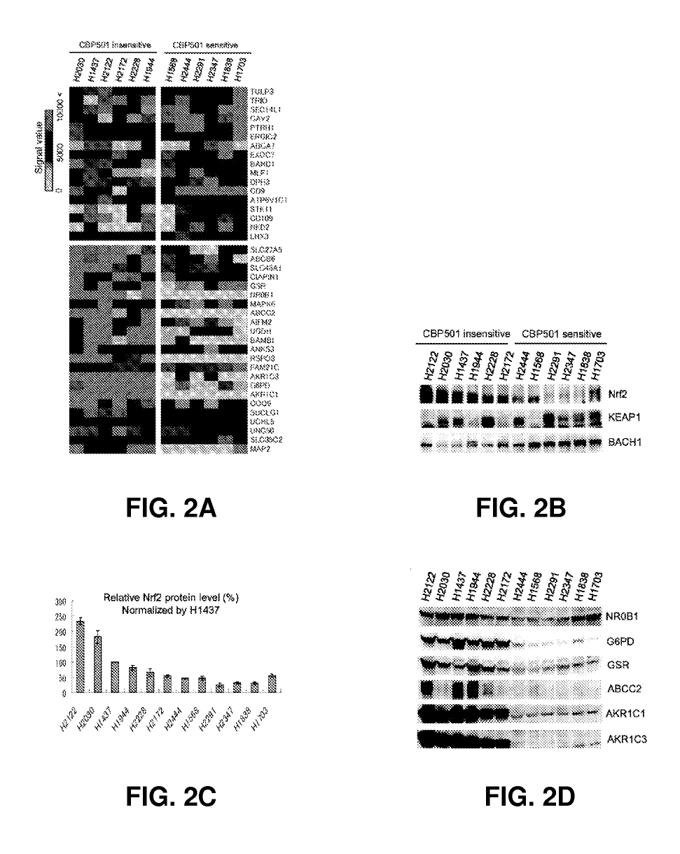

Comprehensive gene expression (Microarray) analysis was performed on the twelve NSCLC cell lines listed in Table 1. Upon setting the threshold signal value for the expression level to be 5000, forty genes showed more than seventy percent of correlation with CBP501 sensitivity (FIG. 2A). These identified genes were subdivided into two categories: genes highly expressed in sensitive cell lines or genes highly expressed insensitive cell lines. Examination of the average expression level values for groupings of genes individually classified as sensitive or insensitive indicated that some groupings of insensitive genes consistently exhibited discernable differences in expression level when CBP501 sensitive and insensitive cell lines were compared (FIG. 7). Western blot analysis was also performed on the twelve NSCLC cell lines listed in Table 1 for over forty proteins that act in a variety of biological processes including stress response, drug resistance, metabolism, differentiation and antioxidant response (data not shown). The expression level of Nrf2 protein was found to correlate well with CBP501 sensitivity in the same set of NSCLC cell lines (FIG. 2B, C). Based on these analyses, focused was placed on the mRNA expression levels of Glutathione reductase (GSR), Glucose-6-phosphate dehydrogenase (G6PD), ATP-binding cassette sub-familyC member2 (ABCC2), Aldo-keto reductase familylC1 (AKR1C1) and AKR1C3 from FIG. 2A because expression of these genes is known to be regulated by a common transcription factor Nrf2 (16-20). It was confirmed that the levels of protein expression correlated with mRNA expression by Western blot analysis (FIG. 2D).

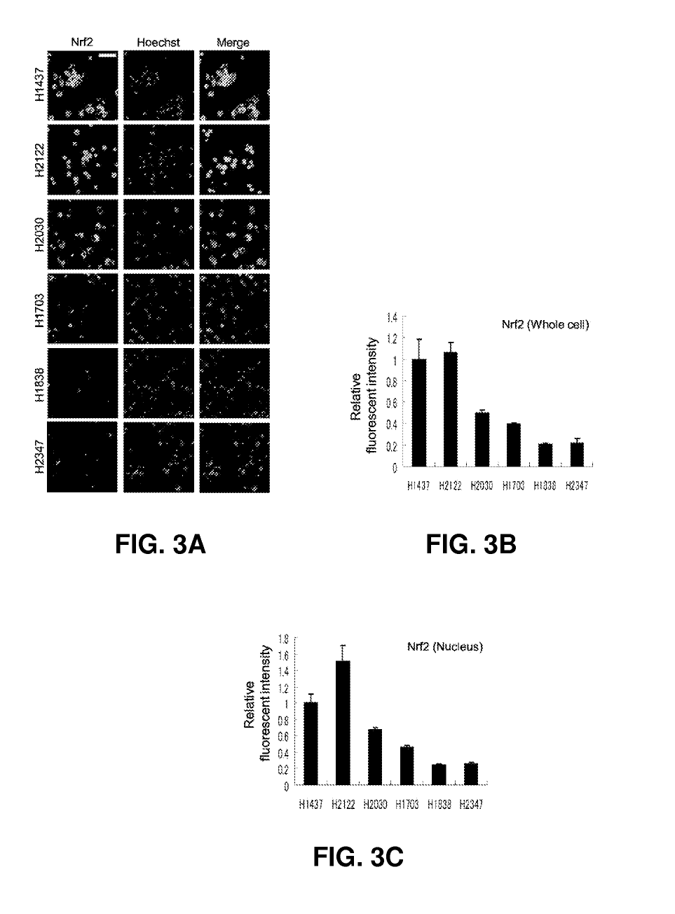

Immunocytochemistry was used to examine intra-cellar localization of Nrf2 in several NSCLC cell lines. These tests revealed both a high degree of nuclear localization for Nrf2 as well as a significantly higher level of whole cell expression for Nrf2 in CBP501 insensitive cell lines compared to CBP501 sensitive cell lines (FIG. 3). These results indicated that high levels of expression of Nrf2 protein in CBP501-insensitive cell lines result in high levels of target gene expression.

CBP501 Sensitivity is Affected by the Availability of Nrf2

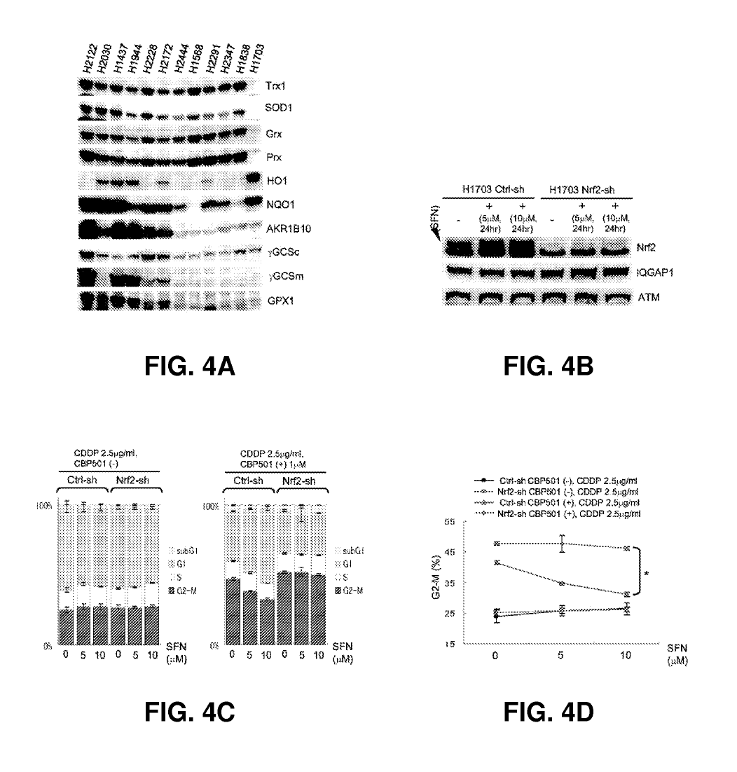

Western blot analyses were performed to examine the expression levels of additional known gene targets for Nrf2 (16-20). NAD(P)H dehydrogenase, quinone1 (NQO1), AKR1B10, γ-glutamyl cysteine synthetase modifier subunit (γGCSm) and Glutathione peroxidase1 (GPX1) each showed higher levels of expression in CBP501-insensitive cell lines (FIG. 4A). These results support that expression of Nrf2 target genes is enhanced in CBP501-insensitive cell lines under normal culture conditions.

In order to demonstrate a direct causal relationship between CBP501 sensitivity and Nrf2 expression, a stable Nrf2 knock-down cell line of H1703 which is sensitive to CBP501 was produced (FIG. 4B). The triple exposure of this cell line to CBP501, CDDP and SFN, a known Nrf2 activator (17) was studied. As shown in FIG. 4B, SFN caused Nrf2 protein levels to increase in a dose dependent manner in the cells transfected with a control shRNA. SFN did not cause a similar increase the levels of Nrf2 protein in cells transfected with Nrf2 shRNA. Addition of SFN attenuated CBP501's effect in cells transfected with control shRNA (FIG. 4C, D). However, the effect of SFN was abrogated in the Nrf2 knock-down strain (FIG. 4B-D).

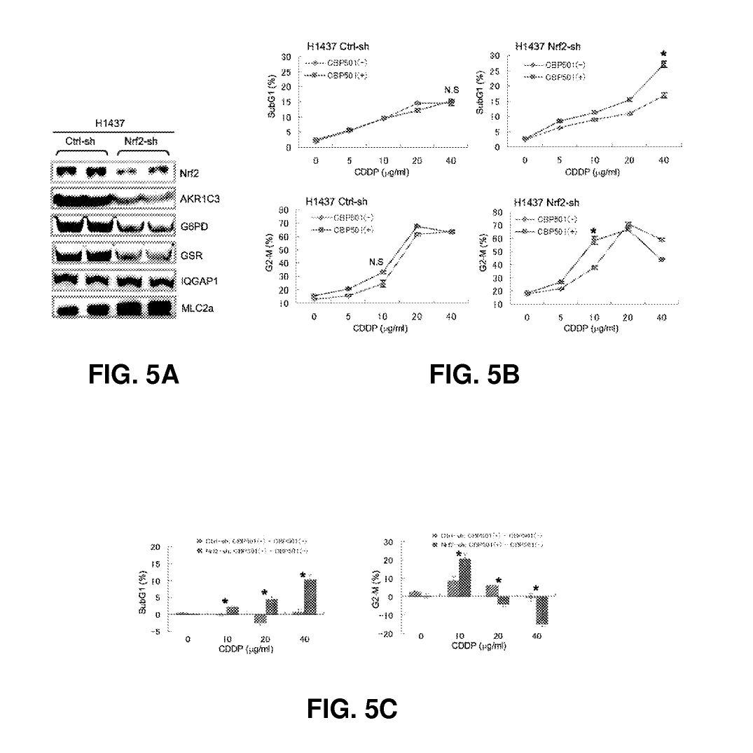

The effect of Nrf2 knock-down in the CBP501 insensitive cell line H1437 was investigated. Nrf2 knock-down was found to cause reduced levels of expression for AKR1C3, G6PD and GSR (FIG. 5A). For this knock-down cell line, CBP501 was found to increase the effects of CDDP on changes in the cell cycle distribution of subG1 and G2-M, reversing the initial CBP501-insensitivity. On the other hand, a cell line transfected with control shRNA showed no difference in CDDP activity with or without CBP501 (FIG. 5B, C). These results indicate that high Nrf2 expression levels might induce resistance to CBP501.

The high expression of Nrf2 protein is a candidate marker to predict resistance to CBP501; Nrf2 targets, at either the protein or mRNA level, are additional candidate markers

Although the expression of Nrf2 or its several downstream transcription targets may possibly predict resistance to CBP501, the general reliability of these predictive markers for CBP501-sensitivity remained to be established. The combination effect of CBP501 and CDDP in sixteen additional NSCLC cell lines, classifying each as CBP501 sensitive or insensitive by the prior criteria was analyzed (Table 2). Classification of CBP501 sensitivity in additional NSCLC cell lines.

TABLE 2

| | Folds increase of CDDP | Definition of CBP501 |
|---|---|---|
| Cell line's name | effect by CBP501 | sensitivity |
| NCI-H1755 | 1 | Insensitive |
| NCI-H358 | 1 | Insensitive |
| NCI-H727 | 1 | Insensitive |
| NCI-H1563 | 1.5 | Insensitive |
| NCI-H647 | 1.5 | Insensitive |
| NCI-H1155 | 1.5 | Insensitive |
| NCI-H520 | 2 | Sensitive |
| A549 | 2 | Sensitive |
| NCI-H1993 | 2 | Sensitive |
| NCI-H1734 | 2 | Sensitive |
| NCI-H522 | 2 | Sensitive |
| NCI-H838 | 2 | Sensitive |
| NCI-H1975 | 2 | Sensitive |
| HCC827 | 4 | Sensitive |
| NCI-H23 | 4 | Sensitive |
| NCI-H1299 | 8 | Sensitive |

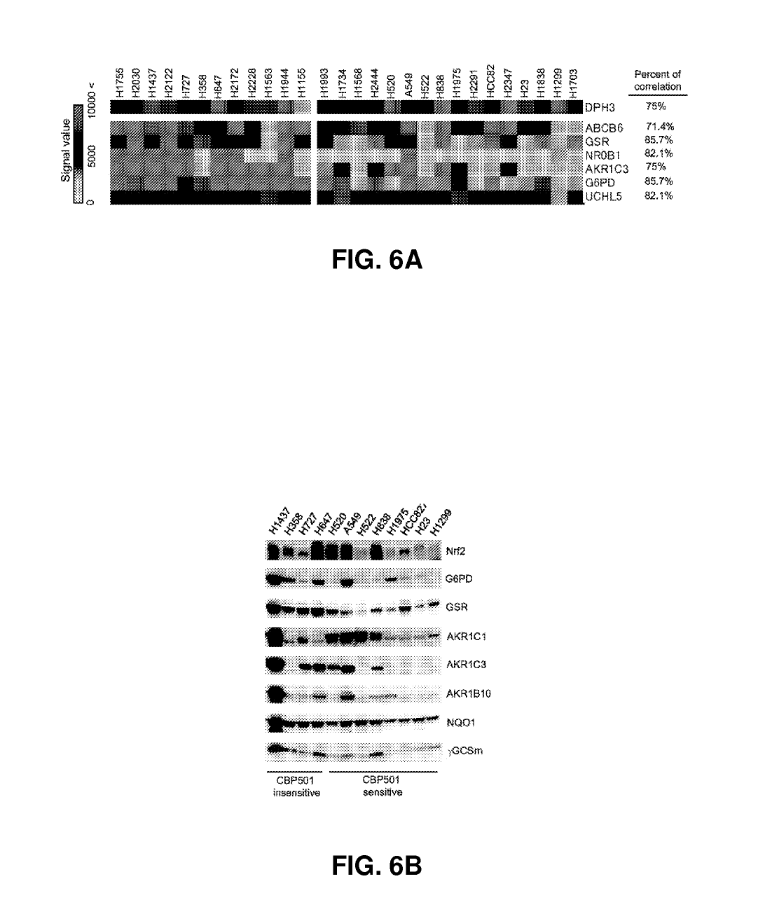

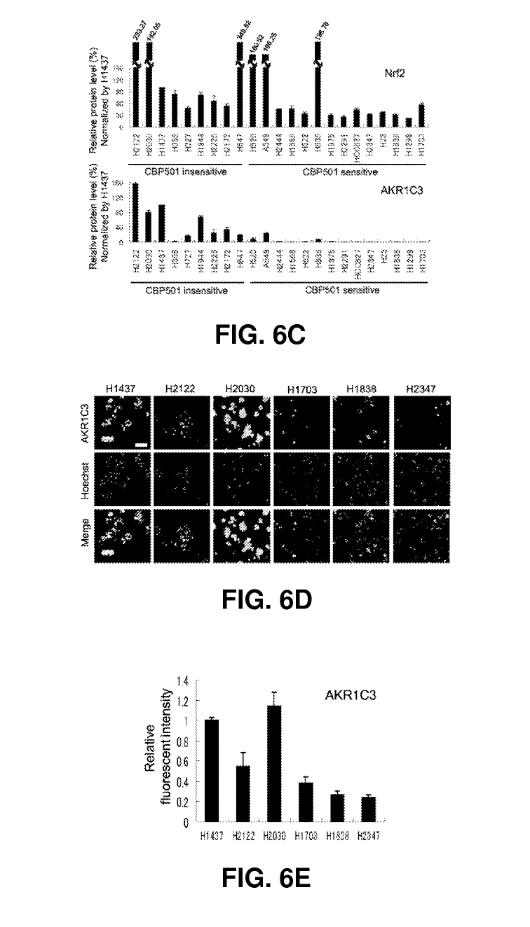

These cell lines were then subjected to the same Microarray analysis as for the initial twelve cell lines. Gene expression heat maps for the expanded set of twenty eight cell lines were again analyzed to identify prediction marker candidates for CBP501 sensitivity having more than seventy percent accuracy. Seven such genes, including three Nrf2 target genes GSR (85.7% prediction rate), AKR1C3 (75%) and G6PD (85.7%), were identified (FIG. 6A). Expression levels of the corresponding proteins in these cell lines were also confirmed by Western blotting (FIG. 6B). Notably, the high expression levels of AKR1C3 and GSR remained comparably correlated with resistance to CBP501 and Nrf2 expression (FIG. 6B, C). To investigate the practical utility of this observation, several NSCLC cell lines were examined to determine whether detection of protein expression of AKR1C3 by immunocytochemical methods might serve as a possible predictive marker for CBP501 sensitivity. Such methodology promises to be a generally applicable for marker detection, since it is sufficiently sensitive to detect expressed protein levels in the small tissue samples that are typically available from NSCLC tumor biopsies (21). From these tests, it was concluded that higher levels of AKRIC3 could indeed be detected in CBP501-insensitive cell lines compared with CBP501-sensitive cell lines (FIG. 6D, E) similar to the results with Nrf2 (FIGS. 3 and 6B). These results indicate that immunohistochemical detection of Nrf2 and a series of Nrf2 gene target molecules can be highly reliable markers for predicting resistance to CBP501, at least in vitro.

It has been indicated that CBP501 has at least two mechanisms of action as an anti-cancer drug candidate (8, 9). One of these, G2 checkpoint abrogation, occurs for treatments of longer duration and at higher doses (8). A second anti-cancer activity of CBP501, the enhancement of CDDP uptake through the binding to calmodulin, occurs for treatments of shorter duration and at lower doses (9). In this Example, focus is placed on the latter effect and characterized the CBP501 sensitivity of many different NSCLC cell lines by treating cells for shorter duration with lower doses of CBP501. The detailed molecular mechanism of this secondary anti-cancer activity of CBP501 remains to be elucidated because the CBP501-calmodulin interaction appears to affect several of the numerous channels and transporters implicated in CDDP uptake (10). These ambiguities about CDDP uptake complicated the identification of single CDDP transport pathway most uniquely affected by CBP501. CBP501 sensitivity was predicted by identifying major differences in gene expression profiles between CBP501 sensitive and insensitive cell lines, as determined by microarray analysis. This comprehensive analysis of gene expression initially led to several candidates for predictive markers of CBP501 sensitivity. Increased expression of genes from a common transcriptional pathway regulated by Nrf2 was identified as a possible indicator of insensitivity to CBP501. Nrf2 is known to be a key transcription factor for genes related to cytoprotective function (22-26). Under homeostatic conditions, Nrf2 is maintained at very low intracellular concentration by the proteasomal degradation system through its association with Kelch-like ECH associated protein 1 (Keapl) and the Cu13 E3 ligase (27-30). It was found that Nrf2 protein expression levels were higher in CBP501 insensitive cell lines than in CBP501-sensitive cell lines. However, Keapl expression levels did not correlate as well with sensitivity to CBP501 (FIG. 2B). This result might indicate that the ability of Nrf2 protein to degrade under homeostatic conditions varies among different cell lines. Several reports indicated a relationship between Keap1 mutation and poor prognosis or chemoresistance in NSCLC cell lines and tumours (31-33). Keap1 mutations are present in a series of NSCLC cell lines that were used in this Example (Table 3 below). Keap1 mutations had a tendency to be present in CBP501-insensitive cell lines. This indicates that Keap1 mutation status is a useful indicator of resistance to CBP501. The role of each mutation type will be further verified.

TABLE 3

| | | | | | | | Functional change | |
|---|---|---|---|---|---|---|---|---|
| Cell | | KEAP1 mutation | | Amino acid | Nudeotide | Amino acid | of KEAP1 by | |
| line's name | CBP501 sensitivity | type | Zygosity | change | mutation | position | mutation | References |
| NCI-H2030 | insensitive | Substitution | homo | V->F | G->T | 568 | Unverified | COSMIC database |
| NCI-H1437 | insensitive | Deletion | homo | — | — | — | No expression | 32 |
| | | | | | | | of KEAP1 | |
| NCI-H2122 | insensitive | Deletion | hetero | — | — | 170-204 | Unverified | COSMIC database |
| NCI-H358 | insensitive | No mutation | — | — | — | — | — | 31 |
| NCI-H647 | insensitive | Substitution | homo | G->W | G->T | 523 | Unverified | COSMIC database |
| NCI-H2172 | insensitive | Substitution | homo | G->C | C->A | 430 | Unverified | COSMIC database |
| NCI-H1944 | insensitive | Substitution | homo | R->L | G->T | 272 | Unverified | COSMIC database |
| NCI-H1993 | sensitive | Substitution | hetero | G->S | G->A | 350 | Unverified | 31 |
| A549 | sensitive | Substitution | homo | G->C | G->T | 333 | Loss of function | 31 |
| NCI-H838 | sensitive | Nosence mutation | homo | — | G->T | 443 | Loss of function | 31 |
| NCI-H23 | sensitive | No mutation | — | — | — | — | — | 31 |
| NCI-H1299 | sensitive | No mutation | — | — | — | — | — | 31 |
| NCI-H1703 | sensitive | No mutation | — | — | — | — | — | 31 |

Differential expression of several Nrf2 target genes at the RNA or protein level was identified as a means to identify candidates for markers to predict CBP501 insensitivity. Of these initial candidates, G6PD, GSR and AKR1C3 showed significant differences in both mRNA and protein expression levels between CBP501 sensitive and insensitive cell lines. Regulation of the expression of these identified genes was recognized as being under the common control of Nrf2 binding elements called AREs (antioxidant response elements) (23). Several known transcription factors can antagonize Nrf2 by competing for interaction at AREs. These include the small MAF proteins, BACH1, c-FOS and FRA1 (23, 34, 35). These transcriptional regulators might be involved in controlling the differential expression of different Nrf2 target genes. In fact, BACH1 protein is one for which expression in NSCLC cell lines appears to increase in CBP501 sensitive cell lines (FIG. 2B).

Nrf2 participates in a diverse spectrum of different biological phenomena, including metabolism, the xenobiotic response, and the antioxidant response, by inducing the expression of numerous genes (22-26). For instance, inducing the expression of G6PD and GSR elevates glutathione levels and this leads to the antioxidant response (36, 37). AKR1C3 can mediate pathways leading to the synthesis of testosterone and to prostaglandin formation (38, 39). Although Nrf2 knock-down experiments indicate that Nrf2 might modulate the CBP501 sensitivity, there is still no definitive answer about which Nrf2 target gene or phenomena directly affect CBP501-sensitivity. One or several Nrf2 target genes might be involved in determining cell sensitivity to CBP501's effects on CDDP uptake. Such direct effects might be difficult to detect by conventional microarray analysis which can sometimes be hindered by technical limitations such as variable sensitivity in the detection of individual probes. Although it has been indicated that the CBP501/CDDP combination effect on short term exposure correlated well with CDDP uptake in at least seven cell lines (9), platinum accumulation in NSCLC cell lines was not demonstrated. So there was a possibility that CBP501 affected not only enhancement of CDDP uptake but also inhibition of the Nrf2 dependent cytoprotective effect against CDDP. For example, reduced glutathione (GSH), which can be up-regulated by Nrf2, binds to CDDP and can decrease toxicity (40). However, measured levels of intracellular reduced GSH in NSCLC cell lines do not correlate with CBP501 sensitivity (data not shown). Clearly, further investigation is needed to establish a concrete mechanism by which Nrf2 regulates CBP501 insensitivity.

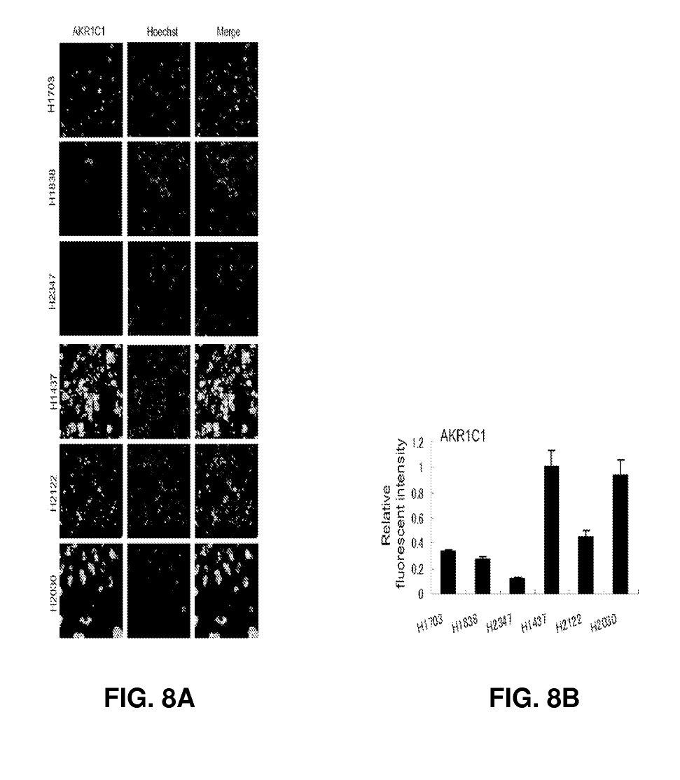

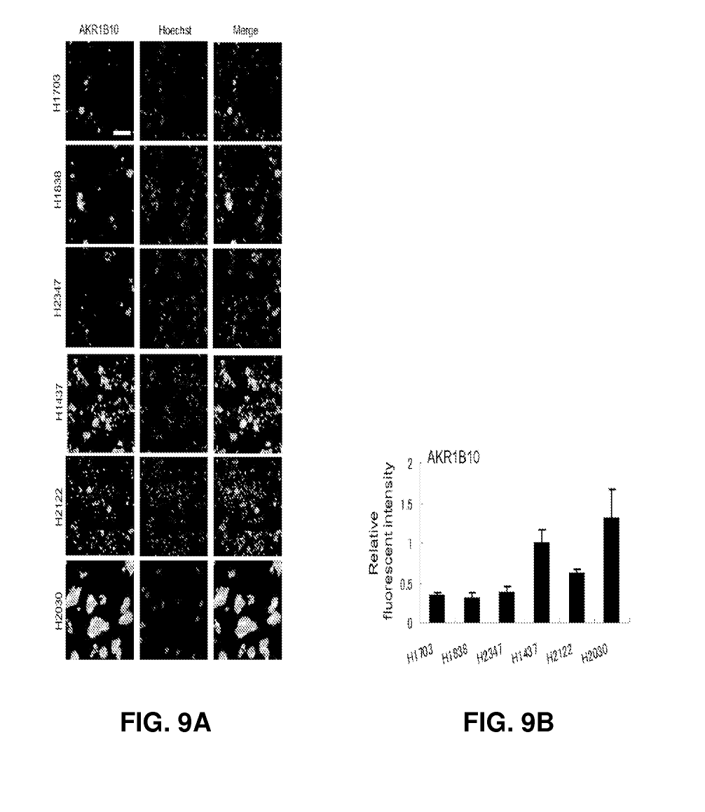

For the actual selection of patients who might be benefited by CBP501 sensitivity, though, immunohistochemistry (IHC) may be a first-line method of choice since it may be possible to obtain a clear indication of sensitivity from the small tumor samples that are available through biopsy. Smaller samples can be analyzed by IHC than by Western blotting (21). Investigations into immunocytochemical measurements for Nrf2, AKR1C1, AKR1B10 and AKR1C3 indicated that they reliably reproduced the results obtained in Western blotting experiments (FIG. 3, 6C, D, FIGS. 8 and 9). Validation of the use of IHC to detect these proteins in tumor biopsy samples and further validation of the correlation with CBP501-sensitivity can provide a useful tool to predict a patient's increased responsiveness to combined CDDP+CBP501 therapy.

### REFERENCES

1. Berardi R, Santoni M, Morgese F, Ballatore Z, Savini A, Onofri A, et al. Novel small molecule EGFR inhibitors as candidate drugs in non-small cell lung cancer. Onco Targets Ther 2013; 6:563-76
2. Koudelakova V, Kneblova M, Trojanec R, Drabek J, Hajduch M. Non-small cell lung cancer - genetic predictors. Biomed Pap Med Fac Univ Palacky Olomouc Czech Repub 2013; 157:125-36
3. Ou S H, Bartlett C H, Mino-Kenudson M, Cui J, Iafrate A J. Crizotinib for the treatment of ALK-rearranged non-small cell lung cancer: a success story to usher in the second decade of molecular targeted therapy in oncology. Oncologist 2012; 17:1351-75
4. Thumar J, Giesen E, Kluger H M. Drug targets and predictive biomarkers in the management of metastatic melanoma. Pharmgenomics Pers Med 2012; 5:139-48
5. Shapiro G I, Tibes R, Gordon M S, Wong B Y, Eder J P, Borad M J, et al. Phase I studies of CBP501, a G2 checkpoint abrogator, as monotherapy and in combination with cisplatin in patients with advanced solid tumors. Clin Cancer Res 2011; 17:3431-42
6. Lee M K, Antoinette J W, Hedy L K, Ronald F, Marianna K, Jose L M, et al. Randomized phase II trial of pemetrexed/cisplatin with or without CBP501 in patients with advanced malignant pleural mesothelioma (MPM). ASCO annual meeting 2012; No.7029
7. Matsumoto Y, Shindo Y, Takakusagi Y, Takakusagi K, Tsukuda S, Kusayanagi T, et al. Screening of a library of T7 phage-displayed peptides identifies alphaC helix in 14-3-3 protein as a CBP501-binding site. Bioorg Med Chem 2011; 19:7049-56
8. Sha S K, Sato T, Kobayashi H, Ishigaki M, Yamamoto S, Sato H, et al. Cell cycle phenotype-based optimization of G2-abrogating peptides yields CBP501 with a unique mechanism of action at the G2 check point. Mol Cancer Ther 2007; 6:147-53
9. Mine N, Yamamoto S, Saito N, Yamazaki S, Suda C, Ishigaki M, et al. CBP501-calmodulin binding contributes to sensitizing tumor cells to cisplatin and bleomycin. Mol Cancer Ther 2011; 10:1929-38
10. Burger H, Loos W J, Eechoute K, Verweij J, Mathijssen R H, Wiemer E A. Drug transporters of platinum-based anticancer agents and their clinical significance. Drug Resist Updat 2011; 14:22-34
11. Hirotsu Y, Katsuoka F, Funayama R, Nagashima T, Nishida Y, Nakayama K, et al. Nrf2-MafG heterodimers contribute globally to antioxidant and metabolic networks. Nucleic Acids Res 2012; 40:10228-39
12. DeNicola G M, Karreth F A, Humpton T J, Gopinathan A, Wei C, Frese K, et al. Oncogene-induced Nrf2 transcription promotes ROS detoxification and tumorigenesis. Nature 2011; 475:106-9
13. Taguchi K, Motohashi H, Yamamoto M. Molecular mechanisms of the Keap1-Nrf2 pathway in stress response and cancer evolution. Genes to Cells 2011; 16:123-40
14. Kawabe T. G2 checkpoint abrogators as anti-cancer drugs. Mol Cancer Ther 2004; 3:513-9
15. Blagosklonny M V, Pardee A B. Exploiting cancer cell cycling for selective protection of normal cells. Cancer Res 2001; 61:4301-5
16. Mahaffey C M, Zhang H, Rinna A, Holland W, Mack P C, Forman H J. Multidrug-resistant protein-3 gene regulation by the transcription factor Nrf2 in human bronchial epithelial and non-small-cell lung carcinoma. Free Radic Biol Med 2009; 46:1650-7
17. MacLeod A K, McMahon M, Plummer S M, Higgins L G, Penning T M, Igarashi K, et al. Characterization of the cancer chemopreventive NRF2-dependent gene battery in human keratinocytes: demonstration that the KEAP1-NRF2 pathway, and not the BACH1-NRF2 pathway, controls cytoprotection against electrophiles as well as redox-cycling compounds. Carcinogenesis 2009; 30:1571-80
18. Aleksunes L M, Goedken M J, Rockwell C E, Thomale J, Manautou J E, Klaassen C D. Transcriptional regulation of renal cytoprotective genes by Nrf2 and its potential use as a therapeutic target to mitigate cisplatin-induced nephrotoxicity. J Pharmacol Exp Ther 2010; 335:2-12
19. Abdullah A, Kitteringham N R, Jenkins R E, Goldring C, Higgins L, Yamamoto M, et al. Analysis of the role of Nrf2 in the expression of liver proteins in mice using two-dimensional gel-based proteomics. Pharmacol Rep 2012; 64:680-97
20. Wu K C, Cui J Y, Klaassen C D. Effect of graded Nrf2 activation on phase-I and -II drug metabolizing enzymes and transporters in mouse liver. PLoS One 2012; 7:e39006
21. Besse B, Olaussen K A, Soria J C. ERCC1 and RRM1: ready for prime time? J Clin Oncol 2013; 31:1050-60
22. Wakabayashi N, Slocum S L, Skoko J J, Shin S, Kensler T W. When NRF2 talks, who's listening? Antioxid Redox Signal 2010; 13:1649-63
23. Hu R, Saw C L, Yu R, Kong A N. Regulation of NF-E2-related factor 2 signaling for cancer chemoprevention: antioxidant coupled with antiinflammatory. Antioxid Redox Signal 2010; 13:1679-98
24. Brigelius-Flohé R, Midler M, Lippmann D, Kipp A P. The yin and yang of nrf2-regulated selenoproteins in carcinogenesis. Int J Cell Biol 2012; 2012:486147
25. Ashrafian H, Czibik G, Bellahcene M, Aksentijevié D, Smith A C, Mitchell S J, et al. Fumarate is cardioprotective via activation of the Nrf2 antioxidant pathway. Cell Metab 2012; 15:361-71
26. Mitsuishi Y, Taguchi K, Kawatani Y, Shibata T, Nukiwa T, Aburatani H, et al. Nrf2 redirects glucose and glutamine into anabolic pathways in metabolic reprogramming Cancer Cell 2012; 22:66-79
27. Itoh K, Wakabayashi N, Katoh Y, Ishii T, Igarashi K, Engel J D, et al. Keap1 represses nuclear activation of antioxidant responsive elements by Nrf2 through binding to the amino-terminal Neh2 domain. Genes Dev 1999; 13:76-86
28. Cullinan S B, Gordan J D, Jin J, Harper J W, Diehl J A. The Keap1-BTB protein is an adaptor that bridges Nrf2 to a Cul3-based E3 ligase: oxidative stress sensing by a Cul3-Keap1 ligase. Mol Cell Biol 2004; 24:8477-86
29. Kobayashi A, Kang M I, Okawa H, Ohtsuji M, Zenke Y, Chiba T, et al. Oxidative stress sensor Keap1 functions as an adaptor for Cul3-based E3 ligase to regulate proteasomal degradation of Nrf2. Mol Cell Biol 2004; 24:7130-9
30. Zhang D D, Lo S C, Cross J V, Templeton D J, Hannink M. Keapl is a redox-regulated substrate adaptor protein for a Cul3-dependent ubiquitin ligase complex. Mol Cell Biol 2004; 24:10941-53
31. Singh A, Misra V, Thimmulappa R K, Lee H, Ames S, Hoque M O, et al. Dysfunctional KEAP1-NRF2 interaction in non-small-cell lung cancer. PLoS Med 2006; 3:e420
32. Kohno T, Otsuka A, Girard L, Sato M, Iwakawa R, Ogiwara H, et al. A catalog of genes homozyugously deleted in human lung cancer and the candidacy of PTPRD as a tumor suppressor gene. Genes Chromosomes Cancer 2010; 49:342-52
33. Sporn M B and Liby K T. NRF2 and cancer: the good, the bad and the importance of context. Nat Rev Cancer 2012; 8:564-71
34. Venugopal R, Jaiswal A K. Nrf1 and Nrf2 positively and c-Fos and Fra1 negatively regulate the human antioxidant response element-mediated expression of NAD(P)H:quinone oxidoreductasel gene. Proc Natl Acad Sci 1996; 93:14960-5
35. Nguyen T, Huang H C, Pickett C B. Transcriptional regulation of the antioxidant response element. Activation by Nrf2 and repression by MafK. J Biol Chem 2000; 275:15466-73
36. Mulquiney P J, Kuchel P W. Model of 2,3-bisphosphoglycerate metabolism in the human erythrocyte based on detailed enzyme kinetic equations: equations and parameter refinement. Biochem J 1999; 342:581-96
37. Coelho P M, Salvador A, Savageau M A. Relating mutant genotype to phenotype via quantitative behavior of the NADPH redox cycle in human erythrocytes. PLoS One 2010; 5:e13031
38. Barski O A, Tipparaju S M, Bhatnagar A. The aldo-keto reductase superfamily and its role in drug metabolism and detoxification. Drug Metab Rev 2008; 40:553-624
39. Byrns M C, Jin Y, Penning T M Inhibitors of type 5 17β-hydroxysteroid dehydrogenase (AKR1C3): overview and structural insights. J Steroid Biochem Mol Biol 2011; 125:95-104
40. Chen H H, Kuo M T. Role of glutathione in the regulation of Cisplatin resistance in cancer chemotherapy. Met Based Drugs 2010; 430939:1-7

1. A method for treating a cancer in a subject, comprising,
a.) measuring expression of nuclear factor erythroid-2 related factor 2 (NRF2), or an NRF2 target gene, in a candidate subject having cancer, or a cancer sample from the candidate subject, and determining the amount of NRF2 in the sample or in the subject having cancer;
b.) comparing the amount of NRF2 determined, or NRF2 target gene determined, to a baseline or reference amount of NRF2 or NRF2 target gene, thereby determining if the amount of NRF2 or NRF2 target gene in the sample or in the subject having cancer is less than the baseline or reference amount of NRF2 or NRF2 target gene; and
c.) treating the subject having the cancer with CBP501 if expression of the NRF2, or the NRF2 target gene in the sample or in the subject having cancer is less than the baseline or reference amount of NRF2 or NRF2 target gene.

2. A method for treating a cancer in a subject, comprising,
a.) screening for a normal or a functional KEAP 1, or a mutation that reduces or decreases activity, function or expression of KEAP 1, in a candidate subject having cancer, or a cancer sample from the candidate subject, and determining the presence of a normal or a functional KEAP 1, or a mutation that reduces or decreases activity, function or expression of KEAP 1; and
b.) treating the subject having the cancer with CBP501 if the subject expresses a normal or a functional KEAP 1, or the subject does not have a mutation that reduces or decreases activity, function or expression of KEAP 1.

3. A method for treating a cancer in a subject with CBP501, comprising,
a.) identifying and/or selecting a subject with a cancer: (i) in which NRF2 expression or NRF2 target gene expression in the subject is less than a baseline or reference amount of NRF2 or NRF2 target gene, or (ii) has normal or a functional KEAP 1 or a mutation that reduces or decreases activity, function or expression of KEAP 1; and
b.) treating the cancer in the subject with CBP501: (i) if the NRF2 or the NRF2 target gene in the sample or in the subject is less than the baseline or reference amount of NRF2 or NRF2 target gene, or (ii) if the subject has normal or a functional KEAP 1 or the subject does not have a mutation that reduces or decreases activity, function or expression of KEAP 1.

4. A method for selecting a subject for cancer treatment with CBP501, comprising,
a.) measuring expression of nuclear factor erythroid-2 related factor 2 (NRF2), or an NRF2 target gene, in a candidate subject having cancer, or a cancer sample from the candidate subject, and determining the amount of NRF2 or NRF2 target gene in the subject or cancer sample;
b.) comparing the amount of NRF2 determined, or NRF2 target gene determined, to a baseline or reference amount of NRF2 or NRF2 target gene, thereby determining if the amount of NRF2 or NRF2 target gene is less than the baseline or reference amount of NRF2 or NRF2 target gene; and
c.) selecting the subject for cancer treatment with CBP501 if the NRF2 or NRF2 target gene in the sample or in the subject is less than the baseline or reference amount of NRF2 or NRF2 target gene.

5. A method for selecting a subject for cancer treatment with CBP501, comprising,
a.) measuring screening for a normal or a functional KEAP 1 or a mutation that reduces or decreases activity, function or expression of KEAP 1; and
b.) selecting the subject for cancer treatment with CBP501 if the subject has a normal or a functional KEAP 1, or the subject lacks a mutation that reduces or decreases activity, function or expression of KEAP 1.

6. A method for identifying a candidate subject for cancer treatment with CBP501, comprising,
a.) measuring expression of nuclear factor erythroid-2 related factor 2 (NRF2) or NRF2 target gene in a candidate subject having cancer, or a cancer sample from the candidate subject, and determining the amount of NRF2 or NRF2 target gene in the subject or cancer sample;
b.) comparing the amount of NRF2 determined, or NRF2 target gene determined, to a baseline or reference amount of NRF2 in order to determine if the amount of NRF2 or NRF2 target gene is less than the baseline or reference amount of NRF2 or NRF2 target gene; and
c.) identifying the subject as a candidate for cancer treatment with CBP501 if the NRF2 or NRF2 target gene in the sample or in the subject is less than the baseline or reference amount of NRF2 or NRF2 target gene.

7. A method for identifying a candidate subject for cancer treatment with CBP501, comprising,
a.) screening for a mutation that reduces or decreases activity, function or expression of KEAP 1; and
b.) identifying the subject as a candidate for cancer treatment with CBP501 if the subject has a normal or a functional KEAP 1, or if the subject lacks a mutation that reduces or decreases activity, function or expression of KEAP 1.

8. A method for characterizing a cancer as more responsive or less responsive to treatment with CBP501, comprising,
a.) measuring expression of nuclear factor erythroid-2 related factor 2 (NRF2) or NRF2 target gene of cells of a cancer, and determining the amount of NRF2 or NRF2 target gene expressed;
b.) comparing the amount of NRF2 determined, or NRF2 target gene determined, to a predetermined value for NRF2 or NRF2 target gene in order to determine if the amount of NRF2 or NRF2 target gene is less or greater than the predetermined value for NRF2 or NRF2 target gene; and
c.) characterizing the cancer as more responsive or less responsive to treatment with CBP501 if the NRF2 or NRF2 target gene expression is less than or greater than the predetermined value for NRF2 or NRF2 target gene.

9. A method for characterizing a cancer as more responsive to treatment with CBP501, comprising,
a.) screening for a normal or a functional KEAP 1 or a mutation that reduces or decreases activity, function or expression of KEAP 1;
b.) characterizing the cancer as more responsive to treatment with CBP501 if a normal or a functional KEAP 1 is present, or a mutation that reduces or decreases activity, function or expression of KEAP 1 is absent or not present.

10. The method of any of claims 1-3, wherein the NRF2 target gene is any of Glutathione reductase (GSR), Glucose-6-phosphate dehydrogenase (G6PD), ATP-binding cassette sub-familyC member2 (ABCC2), Aldo-keto reductase family1C1 (AKR1C1), Aldo-keto reductase familylC3 (AKR1C3), NAD(P)H dehydrogenase, quinonel (NQO1), AKR1B10, γ-glutamyl cysteine synthetase modifier subunit (γGCSm) or Glutathione peroxidase l (GPX 1).

11. The method of any of claim 1-3, wherein the baseline or reference level or predetermined value is determined by expression in cancer cells responsive to CBP501 treatment compared to cancer cells less-responsive to CBP501 treatment.

12. The method of any of claims 1-3, wherein the sample comprises a biological sample.

13.-14. (canceled)

15. The method of any of claims 1-3, wherein the subject is a mammal.

16. The method of any of claims 1-3, wherein the subject is a human.

17. The method of any of claims 1-3, wherein expression is measured by a quantitative assay.

18. The method of any of claims 1-3, wherein expression is measured or detection is by contact with an analyte that detects the NRF2 protein, or the protein encoded by the NRF2 target gene, or detects the mutation that reduces or decreases activity, function or expression of KEAP 1.

19. The method of any of claims 1-3, wherein expression is measured or detection by contact with an analyte that detects the NRF2 transcript, or the transcript of the NRF2 target gene, or by sequencing a nucleic acid that comprises the mutation that reduces or decreases activity, function or expression of KEAP 1.

20. The method of any of claims 1-3, wherein expression is measured or detection is by an immunoassay.

21. (canceled)

22. The method of any of claims 1-3, wherein expression is measured or detection is by a Western blot, ELISA, Northern blot, immunohistochemistry or immunocyotchemistry.

23. The method of any of claims 1-3, wherein expression is measured or detection is by determining cDNA of NRF2 or cDNA of NRF2 target gene.

24. The method of any of claims 1-3, wherein expression is measured or detection is by reverse transcription of NRF2 RNA or NRF2 target gene RNA and polymerase chain reaction (RT-PCR) of NRF2 cDNA or NRF2 target gene cDNA, or reverse transcription of KEAP 1 RNA or KEAP 1 gene and polymerase chain reaction (RT-PCR) of KEAP 1 cDNA.

25. The method of any of claims 1-3, wherein the CBP501 comprises a salt of pro-drug thereof.

26. The method of any of claims 1-3, wherein the CBP501 salt comprises a sodium, calcium, magnesium, nitrate, potassium, phosphate, sulfonate, fumarate, citrate, carbonate, ascorbate, succinate, trifluoroacetate or acetate salt.

27. (canceled)

28. The method of any of claims 1-3, further comprising administering a calmodulin binding agent.

29. The method of any of claims 1-3, further comprising administering a nucleic acid damaging agent to the subject.

30. The method of any of claims 1-3, wherein the nucleic acid damaging agent comprises a molecule that binds to or intercalates in DNA.

31. (canceled)

32. The method of any of claims 1-3, wherein the cancer comprises a lung cancer (NSCLC).

33.-49. (canceled)
